(19) United States
(12) Patent Application Publication
(10) Pub. No.: US 2004/0219186 A1
(43) Pub. Date: Nov. 4, 2004

(54) Expandable gastric retention device

(76) Inventor: James W. Ayres, Corvallis, OR (US)

Correspondence Address: KLARQUIST SPARKMAN, LLP, 121 SW SALMON STREET, SUITE 1600, PORTLAND, OR, 97204, US

(21) Appl. No.: 10/778,917

(22) Filed: Feb. 13, 2004

Related U.S. Application Data
(63) Continuation-in-part of application No. PCT/US01/46146, filed on Oct. 22, 2001.
(60) Provisional application No. 60/313,078, filed on Aug. 16, 2001.

(51) Int. Cl. A61K 9/48; A61F 2/00

(52) U.S. Cl. 424/426; 424/452

(57) ABSTRACT

The present application concerns gastric retention devices formed from compositions comprising polymeric materials, such as polysaccharides, and optional additional materials including excipients, therapeutics, and diagnostics, that reside in the stomach for a controlled and prolonged period of time.

### CROSS REFERENCE TO RELATED APPLICATION

This application is a continuation-in-part of pending International Application No. PCTIUS01/46146, filed Oct. 22, 2001, which claims the benefit of the earlier filing date of U.S. provisional patent application No. 60/313,078, filed Aug. 16, 2001, now abandoned. Both of these prior applications are incorporated herein by reference.

### FIELD

The present application concerns gastric retention devices, formed from compositions comprising polymeric materials, such as polysaccharides, and optional additional materials including excipients, therapeutics, and diagnostics, that reside in the stomach for a controlled and prolonged period of time.

### BACKGROUND

Recent oral drug delivery systems can control drug release in a predetermined manner for a period of time ranging from a few hours to more than 24 hours. The effects of drug therapy depend not only on the drug release pattern from the formulation, however, but also on the kinetics of drug absorption from the gastrointestinal tract. Some drugs are absorbed only in certain regions of the small intestine called “windows of absorption.” Once such drugs pass this region, very little or no drug absorption takes place. Accordingly, there is significant interest in the development of a gastric retention device (GRD) that retains drugs in the stomach for a prolonged and predictable period of time.

In medical care, the timing of drug administration relative to ingestion of food is very important. If a sustained release medication is administered after a meal, the migrating myoelectric complex is interrupted by the food and the dosage form may remain in the stomach for 12 hours or more, which provides an opportunity for drug to be absorbed. However, if the product is administered on an empty stomach, it may empty into the intestine in as little as 20 minutes and be transported through the small intestine in less than 3-5 hours. This can result in dramatically decreased drug absorption for drugs with an absorption window or drugs that are not absorbed if they are not well dissolved in gastric fluid before transfer into the small intestine. Thus, the same medication will produce quite different results depending on whether the medication is taken on a fed or fasted stomach.

The need for a device that can deliver drugs in the stomach for a prolonged, predictable time is well discussed in both the patent and scientific literature, including U.S. Pat. No. 5,651,985 and references therein. Three primary approaches have been utilized in attempts to produce gastric retention devices, and all have suffered from major drawbacks or failures as generally described in U.S. Pat. No. 5,651,985 and a review by Hwang, et al. [Gastric Retentive Drug-Delivery Systems, Critical Reviews in Therapeutic Drug Carrier Systems, 15 (3): 243-284 (1998)]. The most common approach is known as the hydrodynamically balanced (HBS) system (U.S. Pat. Nos. 4,140,755 and 4,167,558), which is designed to float on the contents of the stomach and remain far from the pyloric region of the stomach that empties into the intestine. However, these devices can only float in the stomach if the stomach contains food. For fasting subjects, HBS-type drug dosage forms leave the stomach within a short time. They are swept out of the stomach by the “housekeeping wave,” which is also called the interdigestive myoelectric complex (IMC) or migrating myoelectric complex (MMC). The housekeeping wave has the function of clearing the stomach of undigested materials and is the action responsible for sweeping nickels, quarters, and other ingested solids out of the stomach once any food present is digested and gone.

A second approach to gastric retention devices involves tablets that swell in gastric fluid, as described in U.S. Pat. Nos. 3,574,820 and 4,434,153. Unfortunately, these tablets fall apart when hydrated. The dimensional stability of the materials used to produce swelling tablets greatly decreases with swelling, which leads to premature erosion or dissolution of the gel layer. Further, neither swelling tablets or hydrodynamically balanced systems can incorporate a pre-existing tablet.

A third approach to gastric retention devices involves mechanical operations, such as a polymer envelope that is expanded by the release of a gas after swallowing (see, for example, U.S. Pat. No. 4,207,890). Alternatively, the device can function via the opening of a “flower” structure (U.S. Pat. No. 4,767,627), the unfurling of a rolled up sheet (U.S. Pat. No. 4,308,250 for veterinary use), or via a self-actuated valve with a propellant and a collapsed bag that is converted to a balloon. Expansion of the balloon causes the device to be retained in the stomach (U.S. Pat. No. 3,797,492). Unfortunately, these approaches have not performed well in humans. In particular, a GRD needs to remain in a fasting stomach during times of the MMC, collapse or disintegrate after a predetermined time in the stomach, and it should not prevent the passage of food out of the stomach through the pylorus while the device is in place and food is present. No device has satisfied all of these criteria.

In addition to the approaches outlined above, GRDs have been made from a new category of synthetic acrylamide/sulfopropyl acrylate/acrylic acid polymers containing croscarmellose sodium, also known as “superporous hydrogel composites” (Chen, et al., “Gastric retention properties of superporous hydrogel composites”, Journal of Controlled Release 64, 39-51 (2000); Hwang, et al.). Dried hydrogels typically perform poorly because swelling, especially in sizes that people can swallow (tablets and capsule size made from 1.36 g of starting materials), takes a few hours and may be emptied from the stomach before reaching a fully swollen state. Additionally, even after swelling, the hydrogel is not large enough to prevent the expanded device from passing through the pylorus over an extended period; Chen et al.'s GRD traveled to the colon in only three hours when administered to fasted dogs. In addition, these new polymers do not have FDA or any other governmental regulatory approval.

A further problem with existing GRDs is that, when they do remain in the stomach, they interfere with food transit through the stomach and into the intestine. Apparently, no device is known that will remain in the stomach while still permitting normal food transit.

### SUMMARY

Disclosed herein are GRDs that avoid many of the problems of the prior art because sufficient dimensional stability and flexibility are simultaneously possible in an expandable material that is formed from a mixture comprising a suitable polymer gel. This mixture can be processed to produce a swelling dosage form that is retained in the stomach whether it is administered with or without food. Surprisingly, this composition allows uninterrupted passage of food through the stomach; the device remains in the stomach while the stomach fills and empties normally. The device can be tailored to degrade sufficiently in gastric fluid to leave the stomach in a predetermined time, usually 12-24 hours, but shorter or longer retention times are possible, if desired. Additionally, the gastric retention device is suitable for administration into cavities other than the stomach, for example, oral, rectal, vaginal, nasal, or intestinal cavities. Further, the device can incorporate diagnostic and/or therapeutic agents including, but not limited to, products that already have been formulated and/or marketed, such as solutions, suspensions, emulsions, powders, tablets, capsules, or beads, and can provide gastric retention of the product and controlled release of the drug in the stomach.

The GRDs disclosed herein typically comprise gels formed from a polysaccharide or mixture of polysaccharides. The devices are formed, such as by removing at least a portion of any liquid fraction (e.g. dehydration) followed by compression, to a size suitable for administering to subjects, including humans and animals. Generally, but not necessarily, the formed devices have coatings erodible by gastric fluid applied to an outer surface thereof or are housed within ingestible capsules erodible by gastric fluid. Optionally, the formed devices may have enteric coatings or be housed within enteric capsules. In some embodiments, the polysaccharides comprise carbohydrate gums, and in some embodiments the GRD is formed from a mixture comprising a sugar, a polysaccharide, or combinations thereof. The GRD also can optionally include one or more additional swellable polymers.

The GRD may be processed to form a gel as desired, but described embodiments typically concern thermally induced gels. The GRD may be substantially dehydrated, and in particular embodiments it is freeze-dried. Xanthan gum and locust bean gum are examples of materials used to form working embodiments. When the combination of these two materials is used, the weight ratio of xanthan gum to locust bean gum can vary, for example, from about 1:4 to about 4:1, and in particular embodiments the GRD has a weight ratio of xanthan gum to locust bean gum of from about 1.5:1 to about 1:1. The GRD may further comprise other materials useful for making a dosage form, including, without limitation, a material selected from the group consisting of a plasticizer, a pH adjuster, a GI motility adjuster, a viscosity adjuster, a therapeutic agent, a diagnostic agent, an imaging agent, an expansion agent, a surfactant, and mixtures thereof.

The diagnostic or therapeutic agent can be used as a solution, suspension, emulsion, tablet, capsule, powder, bead, pellet, granules, solid dispersion, or combinations thereof. The diagnostic or therapeutic agent may be more soluble in gastric fluid than intestinal fluid; more soluble in intestinal fluid than gastric fluid; absorbed better within small intestine than within large intestine; absorbed better within stomach than within intestines; and in still other embodiments the diagnostic or therapeutic agent can be absorbed better from the intestines than from the stomach.

In some embodiments the GRD comprises a compressed device that, upon ingestion, expands sufficiently, and is sufficiently robust upon expansion, to preclude passage of the device through a subject's pylorus for a predetermined time up to 24 hours (for example, 2, 6, 9, 12, or 24 hours or more) while still allowing food to pass. The device can be designed to produce virtually any geometric shape upon expansion, such as geometric shape that is substantially a cube, a cone, a cylinder, a pyramid, a sphere, a column, or a parallelepiped. These geometric shapes are generally approximate. For example, the surface of the device typically is not completely smooth.

Generally, the GRD has an expansion coefficient of at least 3.0, and preferably, though not necessarily, the gel expands substantially to 80% of its final size within 2 hours in an aqueous environment, or, optionally, within 2 hours following ingestion by a subject. While not limited to one theory of operation, the expanded gel may have at least one dimension greater than a diameter of a pylorus.

The GRD typically erodes in the presence of gastric fluids and passes through the pylorus after a predetermined time. The GRD may include enzymes that aid erosion of the coating or capsule following ingestion of the device, for example hydrolases, proteases, cellulases, or gluconases.

Disclosed embodiments of the method for making gastric retention devices comprised forming a mixture comprising polymeric materials, processing the mixture to form a dried gel, and optionally coating the dried gel with a material erodible by gastric fluid or placing the gel into a capsule erodible by gastric fluid. Processing may comprise heating the mixture effectively to form a thermally induced gel and freeze-drying the gel. The dried gel may be compressed to a size and shape suitable for administration to a subject prior to coating the gel or placing it in a capsule. The gel can be substantially any geometric shape prior to compression, including for example, a cube, a cone, a cylinder, a pyramid, a sphere, a column, or a parallelepiped, such as a rectangular prism. As noted above, the device may not meet the rigorous geometric definition of these shapes. For example, a device referred to as a parallelepiped may have sides that are not truly parallel.

Also disclosed herein is a method for using gastric retention devices. Embodiments of the method comprise providing a gastric retention device and administering the gastric retention device as generally described herein to a subject. Also disclosed is an embodiment for appetite suppression comprising providing a gastric retention device that expands sufficiently in the stomach of a subject to at least partially suppress appetite in the subject. The gastric retention device is administered periodically to the subject. In some embodiments, the device further comprises an effective amount of a fatty acid, an appetite suppressant, a weight loss agent, or combinations thereof. One aspect of the disclosed method also includes producing a modified pharmacological response without a change in total dose, for example, an increase in urine output with a given oral dose of diuretic.

The foregoing and other features and advantages will become more apparent from the following detailed description of several embodiments, which proceeds with reference to the accompanying figures.

### BRIEF DESCRIPTION OF THE FIGURES

FIG. 1 is a graph of percent hydration in water of xanthan gum/locust bean gum films at various solids ratios.

FIG. 2 is a graph of percent hydration in simulated gastric fluid of xanthan gum/locust bean gum films at various solids ratios.

FIG. 3 is a graph of percent initial hydration in water of xanthan gum/locust bean gum films at various solids ratios.

FIG. 4 is a graph of percent hydration in simulated gastric fluid of xanthan gum/locust bean gum films at various solids ratios during hours 0-3.

FIG. 5 shows the shapes and sizes of four GRDs tested.

FIG. 6 is a graph of the hydration of a GRD in simulated gastric fluid during hours 3-24.

FIG. 7 is a graph of the hydration of a GRD in simulated gastric fluid during hours 0-3.

FIG. 8 is a graph of the amount (milligrams) of amoxicillin released over a 20-hour period from an amoxicillin caplet as compared to an amoxicillin caplet in a GRD.

FIG. 9 is a graph of the amount (milligrams) of amoxicillin released over a 20-hour period from an amoxicillin core caplet as compared to an amoxicillin core caplet in a GRD.

FIG. 10 is a graph of the amount (milligrams) of ranitidine HCl released over a 20-hour period from a Zantac® tablet as compared to a Zantac® tablet in a GRD.

FIG. 11 is a graph of the percent of available riboflavin released over time from riboflavin beads as compared to riboflavin beads in a GRD.

FIG. 12 is a graph of the percent of available riboflavin released over time from riboflavin beads in a modified GRD.

FIG. 13 is a graph of the percent of available riboflavin released over time from a riboflavin solid dispersion in a modified GRD.

FIG. 14 is a digital image of an X-ray view of a fasted dog stomach showing a GRD in the stomach immediately after dosing.

FIG. 15 is a digital image of an X-ray view of a dog stomach showing a GRD in the stomach 2 hours post-dosing.

FIG. 16 is a digital image of an X-ray view of a dog stomach showing a GRD in the stomach 9 hours post-dosing.

FIG. 17 is a digital image of an X-ray view of a dog showing a disintegrated GRD in the colon 24 hours post-dosing.

FIG. 18 is a digital image of an X-ray view of a dog showing a GRD in the stomach 2 hours post-dosing. Food ingested after the GRD was administered has emptied from the stomach while the GRD has not.

FIG. 19 is a digital image of an X-ray of a dog's stomach showing a GRD containing radio-opaque threads. The X-rays show the empty stomach of the dog before dosing, immediately after dosing (0 hr), 1 hour and 2 hours post-dosing.

FIG. 20 is a digital image of an X-ray of a dog's stomach showing a GRD containing radio-opaque threads. The X-rays show the presence of the GRD in the stomach of the dog at 3 hours, 7 hours and 9 hours, and the absence of the GRD at 24 hours post-dosing.

FIG. 21 shows the excretion rate of amoxicillin following administration of an amoxicillin caplet as compared to an amoxicillin caplet in a GRD, both under fasted conditions.

FIG. 22 is a graph showing the excretion rate of amoxicillin following administration of amoxicillin alone as compared to amoxicillin in a GRD under fasted conditions.

FIG. 23 is a graph showing the cumulative amount of riboflavin excreted over time when delivered as an immediate release formulation, or in small, medium, and large GRDs.

FIG. 24 is a graph showing the urinary excretion rate of riboflavin when delivered as an immediate release formulation, or in small, medium, and large GRDs.

FIG. 25 is a graph showing the deconvolved input functions from biostudy data for immediate release and GRD formulations of riboflavin.

FIG. 26 is a graph of the cumulative amount of hydrochlorothiazide excreted vs. time following administration of an immediate release formulation of hydrochlorothiazide as compared to hydrochlorothiazide in a GRD.

FIG. 27 is a graph of the excretion rate of hydrochlorothiazide versus time for the immediate release (IR) capsule and for the new formulation (GRD)

FIG. 28 is a comparison of urine production and water-intake and the cumulative amount of urine output from hydrochlorothiazide in both IR and GRD.

### DETAILED DESCRIPTION

### I. Introduction

Unless otherwise explained, all technical and scientific terms used herein have the same meaning as commonly understood by one of ordinary skill in the art to which this invention pertains. The singular terms “a,” “an,” and “the” include plural referents unless context clearly indicates otherwise. Although methods and materials similar or equivalent to those described herein can be used in the practice or testing of the present invention, suitable methods and materials are described below. The described materials, methods, and examples are illustrative only and are not intended to be limiting.

II. Terms

Term definitions are provided solely for the benefit of the reader, and should not be construed to limit the defined terms to any specific examples provided, or to be definitions that would be narrower than accepted by persons of ordinary skill in the art.

Active agent means any therapeutic or diagnostic agent now known or hereinafter discovered that can be formulated as described herein. Examples of therapeutics, without limitation, are listed in U.S. Pat. No. 4,649,043, which is incorporated herein by reference. Additional examples are listed in the American Druggist, p. 21-24 (February, 1995).

Administration to a subject can be by any known means including, but not limited to, orally, vaginally, rectally, nasally, or in the oral cavity.

Controlled release includes timed release, sustained release, pulse release, delayed release and all terms which describe a release pattern other than immediate release.

Diagnostic means, without limitation, a material useful for testing for the presence or absence of a material or disease, and/or a material that enhances tissue or cavity imaging.

An effective amount is an amount of a diagnostic or therapeutic agent that is useful for producing a desired effect.

Erodible means digestible, dissolvable, soluble, enzymatically cleavable, etc., and combinations of such erosion processes. While not meant to be limiting, one way to measure erodibility is to determine the degree of loss of cohesion of a coating, capsule, or GRD in a given period of time, such as 1, 3, 6, 9, 12 or 24 hours, when the coating, capsule, or GRD is exposed to an appropriate aqueous environment, such as simulated gastric fluid, in a United States Pharmacopeia paddle stirring dissolution apparatus operated at 50 rpm. An appropriate aqueous environment can include one or more than one aqueous media, including changes of media during the study, and often will depend on the specific intended use of the GRD as is well known to those skilled in the art.

Expansion coefficients are calculated by dividing the volume of a GRD prior to expansion into the volume of a fully expanded device.

A Gastric Retention Device (GRD) or Gastric Retention Formulation (GRF) is a device that can be administered to a subject either with or without additional materials. The GRD device can be tailored for various body cavities, including stomach (gastric), intestine, oral, rectal, vaginal, or nasal. Most commonly, for gastric delivery, the device is formed to a size suitable for administration to a subject and, following administration, absorbs liquid and expands to a size greater than the administration size, which is tailored to prevent the passage of the device through a pylorus for a predetermined time. For other body cavities, the device forms a size appropriate for the cavity, e.g. for the intestinal cavity, the device is typically administered orally into the gastric cavity and tailored to form a size appropriate for the intestine. Dehydrated polysaccharide gels may be used to make the device. For routes of administration other than oral administration, the GRD does not necessarily, but typically does, absorb liquid.

Hydrophilic gel-forming materials or agents, also referred to as hydrogels, are materials that hydrate in water and exhibit the ability to retain a significant fraction of water within its structure. Hydrogels may be used to make the GRD device. The hydrogels can be non-cross linked or they may be cross-linked with covalent or ionic bonds. The hydrogels can be of plant or animal origin, hydrogels prepared by modifying naturally occurring structures, and synthetic polymeric hydrogels.

Monosaccharides are aldehyde or ketone derivatives of straight-chain polyhydroxy alcohols containing at least three carbon atoms.

Polysaccharides consist of monosaccharides linked together by glycosidic bonds. This term also includes modified or derivatized polysaccharides, as such compounds often have useful modified properties relative to unmodified polysaccharides.

Tablet is a term that is well known in the art, and is used herein to include all compacted, molded, or otherwise formed materials without limitation in terms of sizes or shapes, and all methods of preparation. Thus, as one common example, compressed or molded shapes known as caplets are included.

III. Composition

Generally, the GRD is made by selecting the material or materials useful for forming an expandable gel matrix, generally monomeric or polymeric materials, such as a polysaccharide. Thereafter, additional materials useful for forming a dosage form, including, by way of example, excipients, diagnostic agents, therapeutic agents, imaging agents or combinations thereof, optionally may be selected and used to form the GRD. The selected polymeric material and materials used to form a desired dosage form, such as at least one excipient, and/or diagnostic or therapeutic agents and/or imaging agents are combined with a liquid to produce a mixture, and the mixture is processed to form a gel containing liquid. A portion of the liquid is then removed from the gel to produce a dried product. This product is referred to herein as a dried “film” even though it can retain substantial height after dehydration. Typically, gels dehydrated by drying at higher temperatures in a vacuum oven collapse or shrink in the vertical direction during dehydration to form a final product which is more relatively “flat” then the original gel but may still retain significant height. When the gels are dehydrated by freeze drying, the film remains in substantially the same shape and size as before drying. Thus, the term film as used herein for dehydrated gels includes all such dehydrated gels independent of the amount of flattening that may occur during dehydration.

The gel film, and, optionally, the dehydrated gel film may be compressed to a size suitable for administration. For example, in a particular working embodiment, the GRD gel was prepared from a mixture comprising, by weight, from about 0.1% to about 2.0% xanthan gum, from about 0.1% to about 2.0% locust bean gum, about 5% polyethylene glycol, about 1% sodium lauryl sulfate, about 1% Carbopol by weight, and a biologically effective amount of a therapeutic, with the remainder comprising water. The device was formed to a suitable size for administration to a subject by drying and compressing sufficiently and into a shape suitable for insertion into a gastrically erodible capsule.

The dried gel film may be coated with or encapsulated by a gastrically erodible and/or enteric coating. Following administration the dry gel hydrates or imbibes liquid. Thus, at various stages the gel may contain liquid or be a dry gel. Each of these steps will be discussed in greater detail below.

A. Monomeric or Polymeric Materials useful for Forming GRDs

Disclosed herein are GRDs that are generally formed from a mixture comprising polymeric materials. However, to the extent that monomeric materials form the same polymeric materials, such as forming such polymeric materials in situ, they may be used as well. The polymeric materials may be hydrophilic gel-forming agents. Examples of hydrophilic gel-forming agents, without limitation, include materials like acacia, tragacanth, guar gum, pectin, xanthan gum, locust bean gum, Carbopol® acidic carboxy polymer, hydroxypropyl methyl cellulose, polycarbophil, polyethylene oxide, poly(hydroxyalkyl methacrylate), poly(electrolyte complexes), poly(vinyl acetate) cross-linked with hydrolyzable bonds, water-swellable N-vinyl lactams polysaccharides, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, hydrophilic colloids such as carboxylmethyl cellulose gum or alginate gum, including both non-cross linked and cross linked alginate gums, wherein the cross linked alginate gums may be cross linked with di- or trivalent ions, polyols such as propylene glycol, or other cross linking agents, Cyanamer® polyacrylamides, Good-rite® polyacrylic acid, starch graft copolymers, Aqua-Keeps® acrylate polymer, ester cross linked polyglucan, similar polymeric materials, and combinations thereof. Some of these hydrogels are discussed in U.S. Pat. Nos. 3,640,741, 3,865,108, 3,992,562, 4,002,173, 4,014,335, and 4,207,893. Each of these patent references is incorporated herein by reference. Hydrogels also are discussed in the Handbook of Common Polymers, by Scott and Roff, published by the Chemical Rubber Company, Cleveland, Ohio, which is incorporated herein by reference.

Polysaccharides have been used to form working embodiments of GRDs. Optionally, the GRD may comprise a carbohydrate gum or may be formed from a mixture comprising a sugar, sugars, a polysaccharide, polysaccharides, or combinations thereof. Working embodiments have used xanthan gum and locust bean gum to form the GRD, and have had a weight ratio of xanthan gum to locust bean gum of from about 1:4 to about 4:1. Particular working embodiments of the GRD have had a weight ratio of xanthan gum to locust bean gum of about 1.5:1 to 1:1. Generally, the polysaccharide comprised from about 0.1% to 5% of the starting materials, and more typically comprised from about 1% to 4%, and more typically still from about 1% to about 3%, with most comprising about 1% of the starting ingredients. Percentages are percent of the total ingredients, including the liquid fraction.

B. Excipients

Optionally, the GRDs also can include an excipient, such as a plasticizer, a pH adjuster, a GI motility adjuster, a viscosity adjuster, an expansion agent, a surfactant, or mixtures thereof.

A plasticizer can be added to the composition to increase the plasticity of the mixture to a level suitable for administering to a subject. Plasticizers may be hydroxylated compounds, particularly poly-hydroxylated organic compounds. For example, polyethylene glycol (PEG) is a poly-aliphatic hydroxylated organic compound that has been used in working examples. Persons skilled in the art could substitute other plasticizers, for example glycerin or surface-active materials. Typically, working embodiments have included from about 1% to 8% plasticizer.

A pH adjuster can be added to adjust the pH of the GRD to a desired pH level. For example, it currently is believed that increasing the pH in the area of the GRD increases expansion in the acidic environment of the stomach. PH adjusters also may be used to modify the viscosity of some polymer excipients such as Carbopol. Suitable pH adjusters include buffers, mineral acids or bases, or organic acids or bases. The pH adjuster is optionally a buffer, and in working examples disodium phosphate and sodium phosphate have been used. Other pH adjusters are known to those of skill in the art, and can include, without limitation, hydrochloric acid, sodium hydroxide, potassium hydroxide, organic acids, such as acetic acid, and organic amines, particularly lower (10 carbon atoms or fewer) alkyl amines, such as triethylamine, and combinations thereof.

A viscosity adjuster can be added to adjust viscosity to a viscosity level that permits retention of the GRD in a stomach for a predetermined time. Viscosity adjusters can include, but are not limited to, Carbopol, polyvinyl pyrollidone, alginates, celluloses, gums, hydrogels, and combinations thereof. Working embodiments have included the viscosity adjusters, Carbopol and polyvinyl pyrollidone. Other viscosity adjusters can be selected by those of skill in the art. Typically, working embodiments have included from about 0.25% to 1% Carbopol and/or polyvinyl pyrollodone.

C. Diagnostics and Therapeutics

The GRD also can incorporate a diagnostic or therapeutic agent. Examples of suitable diagnostics or therapeutics without limitation, can be selected from the group consisting of nucleic acids, proteins, naturally occurring organic compounds, synthetic and semi-synthetic compounds, and combinations thereof. More particularly, the diagnostic or therapeutic agent may be an AIDS adjunct agent, alcohol abuse preparation, Alzheimer's disease management agent, amyotrophic lateral sclerosis therapeutic agent, analgesic, anesthetic, antacid, antiarythmic, antibiotic, anticonvulsant, antidepressant, antidiabetic agent, antiemetic, antidote, antifibrosis therapeutic agent, antifungal, antihistamine, antihypertensive, anti-infective agent, antimicrobial, antineoplastic, antipsychotic, antiparkinsonian agent, antirheumatic agent, appetite stimulant, appetite suppressant, biological response modifier, biological, blood modifier, bone metabolism regulator, cardioprotective agent, cardiovascular agent, central nervous system stimulant, cholinesterase inhibitor, contraceptive, cystic fibrosis management agent, deodorant, diagnostic, dietary supplement, diuretic, dopamine receptor agonist, endometriosis management agent, enzyme, erectile dysfumction therapeutic, fatty acid, gastrointestinal agent, Gaucher's disease management agent, gout preparation, homeopathic remedy, hormone, hypercalcemia management agent, hypnotic, hypocalcemia management agent, immunomodulator, immunosuppressive, ion exchange resin, levocarnitine deficiency management agent, mast cell stabilizer, migraine preparation, motion sickness product, multiple sclerosis management agent, muscle relaxant, narcotic detoxification agent, narcotic, nucleoside analog, non-steroidal anti-inflammatory drug, obesity management agent, osteoporosis preparation, oxytocic, parasympatholytic, parasympathomimetic, phosphate binder, porphyria agent, psychotherapeutic agent, radio-opaque agent, psychotropic, sclerosing agent, sedative, sickle cell anemia management agent, smoking cessation aid, steroid, stimulant, sympatholytic, sympathomimetic, Tourette's syndrome agent, tremor preparation, urinary tract agent, vaginal preparation, vasodilator, vertigo agent, weight loss agent, Wilson's disease management agent, and mixtures thereof. Particular examples of such therapeutics and diagnostics include, without limitation, abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, captopril, carvedilol, caspofingin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fexofenadine, fluticasone propionate, furosemide, gancyclovir, hydrochlorothiazide/triamterene, lamivudine, lamotrigine, lithium carbonate, losartan potassium, melphalan, mercaptopurine, mesalazine, metformin, methyldopa, minocycline, mupirocin calcium cream, nabumetone, naratriptan, omeprazole, ondansetron hydrochloride, orli stat (or a pharmaceutically acceptable salt thereof), ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, selegiline, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine, lamivudine, and combinations thereof.

Effective amounts of the diagnostic or therapeutic agent may be incorporated into the GRD in the form of a solution, suspension, emulsion, tablet, capsule, powder, bead, pellet, granules, solid dispersion, or combinations thereof. Optionally, the diagnostic or therapeutic agent may be more soluble in gastric fluid than intestinal fluid, more soluble in intestinal fluid than gastric fluid, better absorbed within small intestine than within large intestine, better absorbed within stomach than within intestines, or better absorbed within intestines than within stomach.

D. Liquids

The polymeric material, excipient, and/or diagnostic or therapeutic agent can be dissolved and/or suspended in any fluid in which they are at least partly soluble. The preferred liquid is water. Other liquids include polar organic compounds, such as alcohols. Generally, liquid makes up the remainder of the mixture after the polymeric materials, diagnostics and/or therapeutics, and excipients are added.

IV. Forming the GRD

Generally, the GRD is made by combining and mixing the selected ingredients, inducing gelation, drying the resulting gel, and optionally encapsulating the resulting dried, formed gel in a coating, such as a gastrically erodible coating. Each of these steps will be described in greater detail below.

A. Mixing

The method for forming the gel mixture comprises combining the selected polymeric material or materials in the appropriate amounts with the desired amount of liquid and mix with stirring. The excipient or excipients and/or the diagnostic or therapeutic agent or agents may be combined directly with the polymeric material, or, optionally, they may be mixed separately and combined with the mixture of polymeric materials later. Existing dosage forms such as capsules or tablets may be added into the polymeric materials just before gelling, or inserted into the gel after it is formed.

B. Gelation

Traditional tablets and capsules containing polysaccharides and other hydrophilic swelling polymers are produced by compressing discrete powders or granules of these materials mixed with discrete powders or granules of other excipients. Such dosage forms do not include a gel. However, in some cases the materials may hydrate and form a gel after exposure to intestinal fluids. One drawback of such conventional dosage forms is that erosion often occurs faster than gelation, which results in removal of the polymer particles from the surface of the dosage form before sufficient cohesion of the particles develops. In contrast, the gastric retention devices disclosed herein exhibit superior cohesion and gastric retention, in part, due to the formation of a gel prior to administration to a subject. Thus, dosage forms disclosed herein comprise a gel, preferably substantially dehydrated for oral administration forms, before exposure to intestinal fluids.

Gelation can be induced by any method known to those skilled in the art, for example, chemical gelation or thermal gelation. Working examples have used thermally induced gelation primarily to avoid using chemical gelling agents. For example, in specific working examples gelation has comprised heating the mixture sufficiently to achieve dissolution of at least a portion of the solid ingredients, for example heating to a temperature of from about 50° to about 100° C., and typically about 80° C., and maintaining the mixture at such temperature until sufficient dissolution occurs to allow subsequent gelation. Heating times are selected by considering times required for sufficient gelation to occur. Typical heating times with working embodiments have been from about 10 minutes to about 30 minutes for small batches, but variable heating times may occur depending on batch size. Following heating, the mixture is generally cooled to induce gelation, thereby forming a gel. In working processes the mixture is typically cooled to about room temperature. Alternatively, gelation can be performed without heating, i.e., at room temperature as is known to those of ordinary skill in the art.

C. Drying

Liquid can be removed from the formed gel to form a dried film by any means known to those skilled in the art, including air-drying, freeze-drying, vacuum-drying, or any other means of drying or dehydration known to those of skill in the art. Some working embodiments have been dehydrated by vacuum drying at room temperature. Other working embodiments were dehydrated by oven drying at a temperature of from about 35° C. to about 75° C. In other embodiments the gel was dehydrated by freeze drying.

Drying or dehydration means that more than 50% of the liquid solvent total is removed, and usually 90% or more of any liquid present is removed. Liquids used in the formulation may remain in the device as desired either because they help the “dried” gel film retain some pliability and strength, or promote swelling, or because there is no need to completely remove them.

D. Compression

Optionally, the dried film may be compressed to a size and shape suitable for administration to a subject prior to coating the GRD or placing it in a capsule. Any means of compression known to those of skill in the art may be used, though in working embodiments, the dried film has been compressed with compression dies, by rolling, or by squeezing or folding the dried film. In certain working examples, the dried film has been compressed in a punch and die, typically using a pressure of from about 500-3000 pounds per square inch. Typically the dried film is compressed to fit in a size 2, 1, 0, 00 or 000 capsule. These capsule sizes have a volume of about 0.37, 0.50, 0.68, 0.95 and 1.37 mL, respectively. Smaller size capsules may be appropriate for delivering the device through the stomach and into the intestine. When the gels are rehydrated in gastric fluid or simulated gastric fluid, they can swell to the same or greater volume as they displaced prior to drying and compression; however the gels usually regain only up to about 80% of their original volume. No particular percentage of original size is required for efficacy. Similarly, no particular minimum size is required for gastric retention. Using the current formulations, gastric retention has been achieved with parallelepiped devices displacing as little as about 2 mL prior to dehydration and compression.

Typically, the uncompressed dried film has a significantly larger volume than the compressed film. For example, without limitation, the uncompressed film can have a volume of from about 1 mL to about 25 mL, and more typically films have a volume of from about 2 mL to about 20 mL prior to compression.

E. Encapsulation

The dehydrated GRD can have coatings erodible by gastric fluid applied to an outer surface by any means known to those skilled in the art, for example spray coating or dip coating, or by insertion into a capsule. Additionally or alternatively, the GRD can have enteric coatings, such as Eudragit® or Opadry®, applied to an outer surface or can be inserted into a capsule. Working embodiments of the GRD were inserted into size 2, 1, 0, 00, or 000 capsules. One of ordinary skill in the art may choose any known means of coating or encapsulating the GRD.

V. Administration

Generally, the GRDs are administered orally. In some embodiments, however, the GRD may be administered into cavities other than the stomach, for example, oral, rectal, vaginal, nasal, or intestinal cavities. The device may be used as an imaging aid by containing a dye or other imaging material and swelling to fill the cavity. Or, the device may be used to deliver a therapeutic or diagnostic agent to the walls of a cavity for local or systemic effect by swelling and releasing materials into the cavity. For example, the device may be placed into a capsule, and the capsule enteric coated so the device is not released into the stomach, but expands in the intestine to come into contact with the intestinal walls. Swelling of the GRD can also serve to retain the GRD in position in the desired cavity. In certain embodiments, the preferable dimensions of the swollen device can differ from a device designed to be retained in the stomach, and often will be much smaller. For example, presence of the device in the intestine may be used to attenuate hunger and suppress appetite; in this embodiment, the desired GRD size typically is smaller than the gastric-use GRD, particularly when multiple doses are given over time. Even smaller dimensions are preferable for the nasal cavity.

The invention is illustrated by the following non-limiting Examples.

### ExampleS

### Example 1

A. This example concerns methods for making GRDs. The listed materials were obtained and processed as stated below.

I. Dry powders of Xanthan Gum (XG, Spectrum Chemical Mfg. Corp., Gardena, Calif.) and Locust Bean Gum (LBG, Sigma Chemicals, St. Louis, Mo.) were mixed intimately and compressed into a round shape tablet.

II. XG and LBG were dissolved in water at 80° C., gelled, dried, and disrupted. A viscous gel was formed and poured into a Petri dish, and dried in an oven. The thick, dried mass was then crushed into powder and the powder was then compressed into tablets.

III. Accurately weighed LBG (0 g-1 g) was added to lOOml water maintained at 70-75° C. with constant stirring. The resulting solution was heated to a temperature of 80-85° C. for the addition of XG, which was added slowly with constant stirring. The highly viscous solution thus prepared was poured into suitable shaped moulds. Upon cooling, the resulting gel was cut into desired sizes. These gels were dried and subjected to hydration studies.

IV. Accurately weighed LBG (0 g- 1 g) was added to 100 ml water at 70-75° C. with constant stirring. To the resulting solution, at 80-85° C., XG was added with constant stirring. lOml of polyethylene glycol (PEG) 400 was added to the resulting mixture that was cooled (gelled), cut into desired sizes, and dried.

V. Various agents (0.5 g-4 g), individually in separate experiments, including sodium bicarbonate (Mallinckrodt, Paris Ky.), tartaric acid, Water-Loc® (Grain Processing Corp., Muscatine Iowa), hydroxypropyl methylcellulose, polyethylene oxide N-80 (Union Carbide Corp. Danbury, Conn.) were incorporated into the gels prior to drying into films to evaluate the effect of the ingredients on rate of hydration in simulated gastric fluid (SGF).

Tablets made by direct compression of powders of XG mixed with LBG as received from the suppliers did not produce cohesively hydrated gels in either water or gastric fluid. In fact, the tablet fell apart when placed in water or gastric fluid.

Dissolution of both the gums in water produces an interaction that causes gelation to occur. Dissolving XG and LBG in water at 80° C. produced a solution, which, upon cooling, produced a gel that dried to produce a film. Gel strength depended on the temperature at which the interaction between two gums occurred, i.e. temperature at which gel was made. Interaction above the Tₘ of XG results in a gel that has better gel strength. Dissolution of gums at 70-75° C., first LBG followed by XG, gives a gel with better gel strength.

Gels thus made were dried in the oven to produce gel films that were then powdered, and the powder was compressed into tablets. Such tablets fell apart when contacting water or simulated gastric fluid; however, individual particles hydrated extensively when in contact with the medium.

B. In other examples, GRDs were made according to the following method:

Materials

The following chemicals were obtained from standard sources as indicated. All chemicals were used as received.

Xanthan gum (XG; spectrum Chemical Mfg. Corp., Gardena, Calif.), Locust bean gum (galactomannan polysaccharide from seeds of Ceratonia Siliqua, Sigma catalogue # G-0753, Sigma Chemicals, St. Louis, Mo.), polyvinyl pyrrolidone (PVP), and riboflavin (Sigma Chemicals, St. Louis, Mo.), sodium lauryl sulphate (SLS; Matheson Coleman & Bell, Cincinnati Ohio), polyethylene glycol 400 (PEG 400) and polyethylene oxide, molecular weight 200,000 (Union Carbide Corp. Danburg, Conn.), microcrystalline cellulose [Avicel, PH 101] (FMC Corp. Newark, Del.). Barium impregnated polyethylene spheres, 1.5 mm in diameter, (BIPS) (Chemstock Animal Health LTD, New Zealand), Radiopaque threads (provided by the veterinary medical school at Oregon State University).

Two types of GRD were prepared: a regular GRD and the modified GRD. The regular GRD was prepared by dissolving LBG (0.5 gm) and XG (0.75 gm) in 100 ml water. The modified GRD was prepared by dissolving PVP (0.5 gm), LBG (0.5 gm), SLS (0.15 gm), and XG (0.75 gm) in 100 ml water (in that order) with constant stirring. Both solutions were heated to a temperature of 85° C. 6 ml of PEG 400 was then added to each of the hot viscous solution. Accurately weighed riboflavin in the form of powder, beads, or solid dispersion was then added to hot viscous solution with constant stirring to produce a homogenous mass.

The highly viscous solution was then poured into suitably shaped moulds, and the resulting gel was left to cool for 4 hours at room temperature and was cut into desired sizes. The cut gels were dried in a vacuum oven at 50° C. for about 16 hours. The process of drying produced flexible films that could be easily shaped by hand and fitted into capsules. The GRD, consisting of a capsule containing the dried gel (film) with drug, was then suitable for use.

Three different size capsules (‘0’, ‘00’, and ‘000’ size) were filled with differently sized GRD containing riboflavin.

The two main ingredients of the described GRDs are XG and LBG. XG and LBG were used in the ratio of 1.5:1 respectively. Increasing the ratio of XG more than 1.5 produced very viscous gels and harder films after drying. It is difficult to prepare solutions containing more than 3% gums because both XG and LBG are high-viscosity colloids. XG was used in higher ratio than LBG as better pH stability is obtained when the colloid ratio favors XG. XG is stable over the entire pH spectrum, whereas LBG is less acid-stable. Since some GRDs are intended to stay in the acidic environment of the stomach for more than 9 hours, higher XG ratio will produce stronger gel after hydration in gastric fluid and the resulting gel will not degrade rapidly. Solutions containing less than 1% gums were less viscous and the dried films were thinner and, when hydrated in simulated gastric fluid, lost their general rigidity and integrity in less than 6 hours. When PVP and SLS were added in an attempt to increase the rate and amount of riboflavin released from the GRD, surprisingly more elastic films were produced when the gels were dried. Further increasing the amount added of PVP and SLS to the gum solution produced very soft films after drying. These soft films, when immersed in SGF, produced weak gels that lost their integrity in SGF in about 4 hours.

The gums' solution was heated to 85° C. The viscosity of the solution drops sharply at this temperature, which allows the viscous solution to be poured into molds. The viscosity increases sharply again at around 40-50° C. as the solution is cooled from 85° C. Addition of therapeutics, diagnostics, or imaging agents as a solution, suspension, powder, tablet, capsule, bead, pellet, granule, emulsion, solid dispersion, or combinations thereof can occur before the gel is fully formed by cooling.

### Example 2

This section concerns methods for drying gels into films.

A. Using the method of gel preparation outlined in Example 1A, section IV, different methods of drying gels into films were used to produce films having differential hydration periods. Methods employed include oven drying at 45° C., drying under vacuum at 35° C.-60 degrees C., and freeze-drying at −20° C.

Gels dried into films in the oven at temperatures higher than 40° C. tended to lose PEG (as expected, because the boiling point of liquid PEG is around 45° C.). Drying at a lower temperature, such as 30-35° C., took more than 24 hours for the gel to dry into films.

When gels were dried in the oven under vacuum at 30° C., loss of PEG was negligible. Drying time was about 12-18 hours.

There was no loss of PEG when the gels were freeze-dried into films. These films were easy to compress to fit into a capsule. Freeze drying involved initially freezing gels at −20° C. for 2 hours, and then subjecting to freeze-drying at −46° C.

B. GRDs made according to Example 1B were dried according to the following methods:

Flexible soft films were obtained when the gels were dried in a vacuum oven at 50-55° C. for about 16-17 hours. Flexible, soft films facilitate easy rolling and fitting into capsules as well as for quick swelling when immersed in SGF. When higher temperatures were tried (60-70° C.) for shorter time (12-15 hours), harder films were obtained that broke more easily when rolled into capsules and did not swell as well when immersed in SGF. When lower temperatures were tried (30-40° C.) it took about 48 hours to obtain dried films and the dried films did not swell as fast as films produced at 50°-55° C.

### Example 3

This section concerns compression of the dried films into sizes suitable for administration.

Having dried the gel of Example 1A, section IV to form the dried film of Example 2A, the dried films were compressed with the help of specially made punches and dies. A series of dies with decreasingly narrow internal diameters were used. A punch pushes the film from one die into the next die, followed by pushing of the film by another punch into the next die. This process takes place in succession until a point is reached where the film is small enough to put into a desired capsule size, such as a ‘000’ capsule. Other size capsules can be used with other size films or caplets.

### Example 4

This section concerns hydration studies performed on GRDs.

A. In some examples, hydration studies were carried out as follows:

Having prepared the gel according to Example 1A, section IV, dried the gel to form a dried film as outlined in Example 2A, and compressed the dried film as shown in Example 3, hydration studies of films made in different shapes and from various ratios of xanthan gum and locust bean gum were carried out in both water and simulated gastric fluid. Hydration studies in simulated gastric fluid were carried out at 37° C. Percent hydration is calculated as:

$$Percent Hydration = \frac{100 * ( Final wt . of film - Initial wt . of film )}{Initial wt . of film}$$

Films that had been cut into different sizes and shapes were hydrated in water or in gastric fluid. Hydration studies also were carried out in diluted gastric fluid (1 part of SGF and 3 parts water) for comparison. Shapes such as circle, star, cube, rectangle, triangle, etc., were studied.

Of all the shapes studied, a cubic shaped gel that had been dried into a somewhat flat, generally rectangular film that was uneven and non-uniform in height, width, and depth was found to have the fastest swelling and maximum volume, and also had greater gel strength. However, based on studies of sizes that would most easily fit into a capsule, a preferred shape was a rectangular gel shape having dimensions of about 4 cm×4 cm×1 cm, prior to drying.

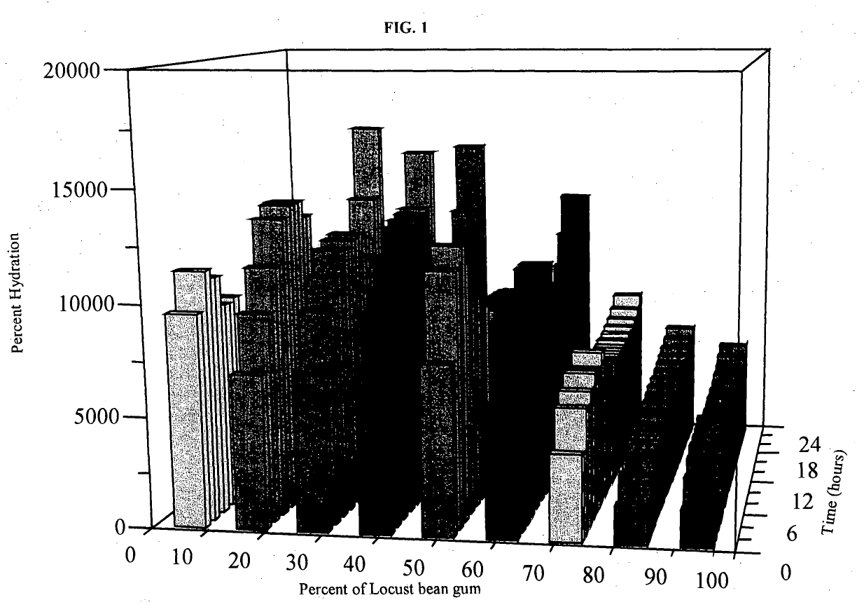

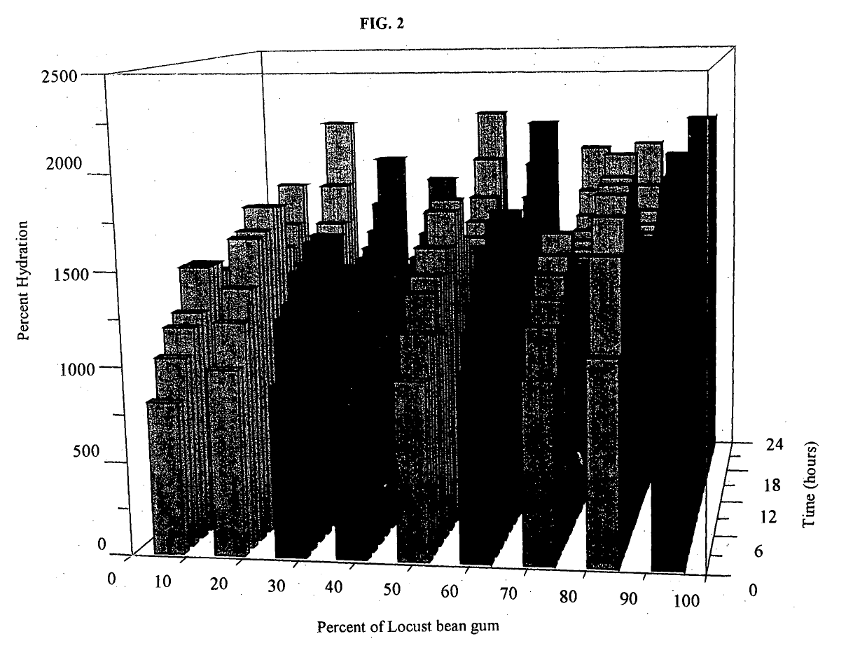

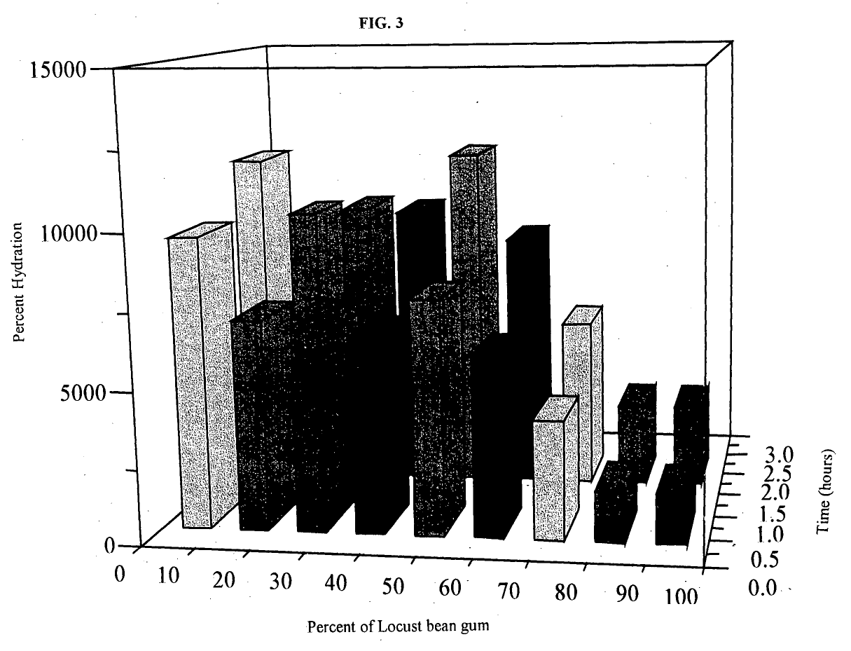

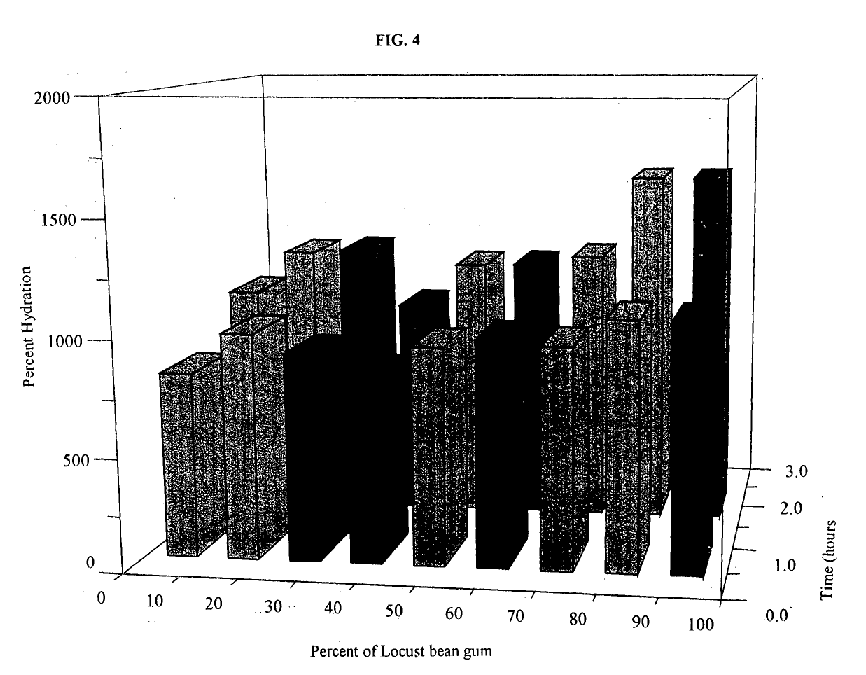

Gels at various solids ratios of xanthan gum to locust bean gum were made as shown in Table 1 and dried into films. Complete hydration of the films in water or simulated gastric fluid for 24 hours is depicted in FIGS. 1 and 2, respectively. Initial hydration of the films in water or simulated gastric fluid is shown in FIGS. 3 and 4, respectively. When a capsule or tablet is ingested on an empty stomach, the time span during which it is passed out of the stomach and into the intestine may range from a few minutes to two hours, depending on the arrival time of MMC (migrating motor complex). A GRD ingested in a capsule should ideally start hydrating as soon as the capsule dissolves and should attain a large enough size within 15-20 minutes to avoid passage through the pyloric sphincter. The structural integrity of the hydrated gel should be sufficient to withstand MMC. Therefore, initial hydration rate and structural integrity are very important.

TABLE 1
Composition of XG/LBG films.

| Film # | XG (% w/w) | LBG (% w/w) |
|---|---|---|
| 1 | 100 | 0 |
| 2 | 90 | 10 |
| 3 | 80 | 20 |
| 4 | 70 | 30 |
| 5 | 60 | 40 |
| 6 | 50 | 50 |
| 7 | 40 | 60 |
| 8 | 30 | 70 |
| 9 | 20 | 80 |
| 10 | 10 | 90 |
| 11 | 0 | 100 |

Based on hydration study profiles and gel strength, a gel with a 50:50 ratio was considered for further modification. Gel strength was based on visual observation during hydration of films and by physical examination of gels formed after film hydration.

As depicted in FIGS. 1 to 4, the hydration of films in SGF is extensive, but comparatively less than that in water. Hydration in water is approximately 10 times greater than in SGF. Hence, in order to make the film swell faster and to a larger size in SGF, addition of the buffering agents disodium phosphate or sodium phosphate was tested. Films containing disodium phosphate or sodium phosphate (twice the amount of gums solids) swell completely in SGF in about 12 hours time. After 12 hours time, the SGF (about 500 ml volume) used for hydration studies was found to have a pH of 6.8. In vivo, there will be continuous secretion of gastric acid with fluids being eliminated from the stomach in a first order process, hence pH in the stomach will not reach 6.8 as it did in vitro, where the volume of acid is fixed. The pH of the microenvironment inside the film as it hydrates, however may remain alkaline or neutral and promote rapid swelling in gastric fluid without changing the pH of the stomach significantly. One limitation to addition of alkalizing agent is that there is a correlation between the amount of the alkalizing agent and the ability to compress the film to fit into a capsule. Hydration of the film in a medium containing 25% simulated gastric fluid and 75% water improved considerably as compared to gastric fluid alone. Medications are ingested with water. Thus, a hydration study carried out in 3:1 water:SGF media simulates the expected conditions when the GRD is ingested with 8-10 ounces of water.

Addition of other additives such as polyethylene oxide, carboxy methylcellulose (CM), and/or Water-Loc® into gels during formation were used to improve the initial hydration of films in SGF. Table 2 depicts various formulations containing different additives. All the above-mentioned studies were evaluated by visual examination of hydration of film after regular intervals of time.

Gels may become too brittle to fold or compress to place inside a capsule. Addition of polyethylene glycol (PEG) into the gel produces more supple films following drying of the gel.

B. In other examples, hydration studies were carried out according to the following method:

Hydration studies on four differently shaped dried gels (films) made of XG, LBG, PVP, SLS, and PEG 400 according to the method of Example 1B were conducted in simulated gastric fluid at 37° C. Dried gels were prepared by dissolving the ingredients in water. The mixture was then heated at 85° C. and 10 ml of the hot viscous solution was poured into different shaped molds to produce the desired shapes. The four shapes were cubic, rectangular, short cylinder, and long cylinder. The gels were then dried and subjected to hydration studies.

TABLE 2
Examples of various formulations studied for
hydration during development of a GRD (percent of total).

| | | | sodium | | | Water- | | | |
|---|---|---|---|---|---|---|---|---|---|
| Form | XG | LBG | alginate | Explotab | PEG | Loc 400 | HCO₃ | NaPO₃ | CM |
| #1 | 0.5 | 0.5 | 0.5 | 1 | PEG300-1 | — | — | — | — |
| #2 | 0.5 | 0.5 | — | 1 | PEG300-1 | — | — | — | — |
| #3 | 0.5 | 0.5 | — | 2 | PEG300-1 | — | — | — | — |
| #4 | 0.5 | 0.5 | — | 1 | PEG400-1 | — | — | — | — |
| #5 | 0.5 | 0.5 | — | 0.5 | PEG400-1 | — | — | — | — |
| #6 | — | — | 4 | 0.5 | PEG300-2 | — | — | — | — |
| #7 | — | — | 4 | 1 | PEG300-2 | — | — | — | — |
| #8 | — | — | 4 | 2 | PEG300-2 | — | — | — | — |
| #9 | 0.5 | 0.5 | — | — | PEG400-5 | — | 1 | — | — |
| #10 | 0.5 | 0.5 | 0.5 | — | Peg400-5 | — | 1 | — | — |
| #11 | 0.5 | 0.5 | — | 1 | PEG400-5 | 1 | — | — | — |
| #12 | 0.5 | 0.5 | — | — | PEG400-5 | 1 | 1 | — | — |
| #13 | 0.5 | 0.5 | — | — | PEG400-5 | — | — | 1 | — |
| #14 | 0.3 | 0.3 | — | 1 | PEG540-5 | — | — | — | 1 |

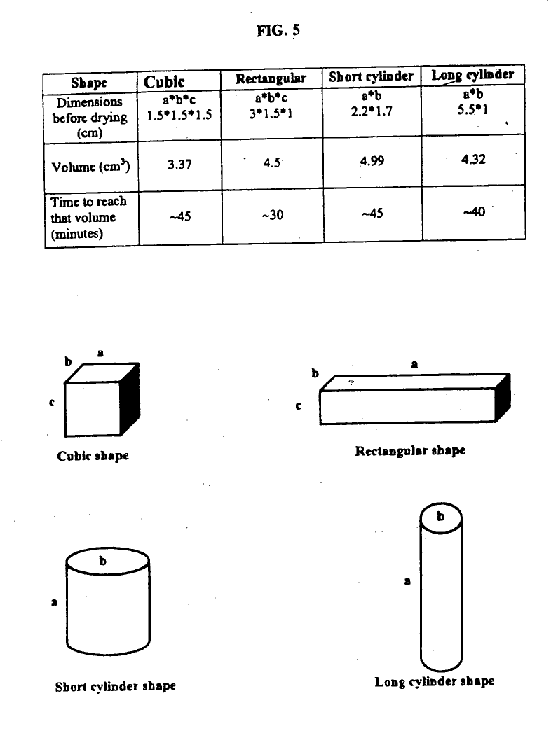

Four different shapes were hydrated in simulated gastric fluid. The dimensions and the shapes of the GRDs examined are shown in FIG. 5. The percent increase in weight of the hydrated films was determined after 15, 30, 45, 60, 120 and 180 minutes and determined again after 12 and 24 hours.

The hydrated films retained their integrity for up to 24 hours in simulated gastric fluid. Of all the shapes studied, a rectangular shaped gel that had been dried into an approximately flat rectangular film was found to have the fastest swelling and maximum volume. Based on this study the rectangular shape was chosen for further in vitro and in vivo studies.

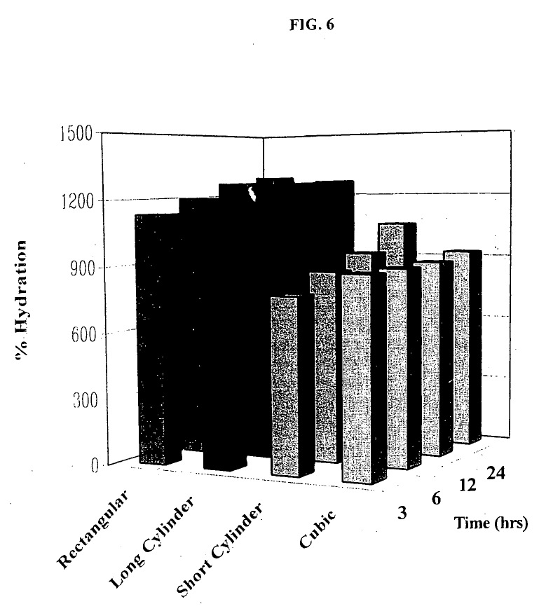

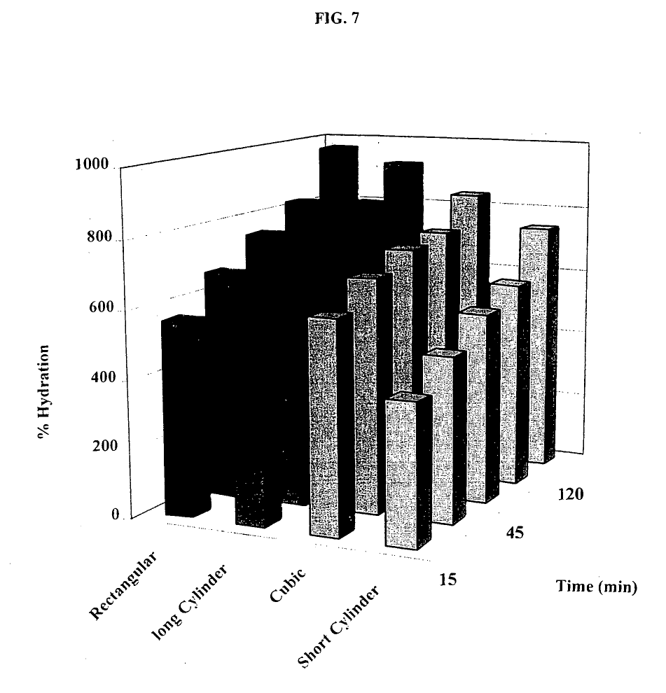

Complete hydration of the films in simulated gastric fluid (SGF) for 24 hr is depicted in FIG. 6. Initial hydration of the films in SGF is shown in FIG. 7. The initial hydration is a very important factor into the development of a GRD. Ideally it is best to make a device that is small enough to fit into a capsule for easy swallowing, but that expands upon contact with gastric juice to a size that is too large to pass through the pylorus. For certain application, the swelling to this large size should be fast (e.g. from aobut 15 to about 30 minutes) to avoid gastric emptying by the strong contractions of the housekeeper wave, which lasts about 5 to 15 minutes. Therefore, fast swelling of the released dried gel and integrity of the swollen gel are very important.

### Example 5

This section concerns methods for incorporating diagnostic or therapeutic agents into GRDs.

A. Amoxicillin was incorporated into the GRD from Example IA, section IV in the form of a tablet with a caplet shape. Amoxicillin was chosen as a model drug because it has a ‘window of absorption’.

The hot viscous solution of gums prepared by the methods described in Example 1A, part IV was poured into suitable moulds so that tablets incorporated into the gel remain suspended.in the gel. This tablet-containing gel was then cut into the desired size. Following drying for 12-18 hours, these dried films containing tablets were compressed in a punch and die with a hydraulic press to fit into a ‘000’ capsule.

B. Riboflavin was incorporated into a GRD from Example 1B in the form of powder, beads, or solid dispersion. Riboflavin incorporated into the gel by stirring into the hot, viscous mixture immediately prior to cooling into a gel, remained suspended in the gel. The dried gels (films) containing drug beads, powder, or solid dispersion were easily rolled and fitted into suitable size capsules. The GRDs containing drug beads, powder, or solid dispersion were then subjected to in vitro dissolution and/or in vivo studies.

### Example 6

This section concerns preparation of amoxicillin caplets and ‘core’ caplets for use with GRDs.

Amoxicillin caplets were prepared by combining the ingredients listed in Table 3 and formed by direct compression.

TABLE 3
Formula for Amoxicillin caplet.

| Ingredients | Quantity (mg) |
|---|---|
| Amoxicillin trihydrate | 287 |
| Avicel PH 112 | 50 |
| Magnesium stearate | 2.5 |

To form the amoxicillin ‘core’ caplets, amoxicillin caplets were centered in a bigger die and punch with microcrystalline cellulose and compressed again such that the amoxicillin caplet is inside the shell formed by microcrystalline cellulose. New caplets thus formed had an amoxicillin caplet as a core, and are commonly known as “core tablets” or a “tablet-within-a-tablet”.

### Example 7

This section concerns preparation of riboflavin formulations for use with GRDs.

Riboflavin was incorporated in the GRD in the form of powder, beads, or solid dispersion. Riboflavin beads were prepared by mixing known amounts of riboflavin, Avicel PH-101, and polyethylene oxide 200,000 with water to produce a wet mass. This mass was then extruded and spheronized using a laboratory extruder (model 10/25) and spheronizer (model 120, Caleva Process LTD, England) to produce drug beads (1.5-2.0 mm in diameter). The beads were left to dry overnight in an oven at 50° C. Beads incorporated into the gel by stirring into the hot, viscous mixture immediately prior to cooling into a gel, remained suspended in the gel.

Riboflavin beads were prepared by extrusion and spheronization using the formula shown in Table 4. Riboflavin when used in powder form was dried for 2 hours at 120° C. before being incorporated into the gel to remove moisture.

TABLE 4
Formula for riboflavin beads

| Ingredients | Quantity (gm) |
|---|---|
| Riboflavin | 70 |
| Avicel PH101 | 25 |
| Polyox (N-80) | 5 |

Riboflavin solid dispersion was prepared by melting a weighed quantity of PEG 3500 in an evaporating dish. A weighed quantity of drug was then added to yield the desired ratio of drug to PEG (1:3). The system was heated until complete dissolution of the drug was achieved. The dish was then transferred to an ice bath and the material stirred until cold. The final solid mass was crushed, pulverized and screened to produce a fine powder. The prepared solid dispersion was dried over night in a vacuum oven at room temperature before being incorporated into gels.

### Example 8

This section concerns dissolution studies carried out on GRDs containing diagnostics and/or therapeutics.

A. This example demonstrates that a therapeutic agent in the form of a tablet can be incorporated into a gastric retention device formed from a polysaccharide, and the device can be formed to a size suitable for administration to a subject, and housed in an ingestible capsule erodible by gastric fluid. Dissolution studies were carried out using GRDs made according to the method of Example 1A, section IV, and containing the model drugs amoxicillin or ranitidine HCl, using the USP XXII paddle method at 37° C. at 75 rpm for 20 hours. Dissolution medium consisted of 900 ml simulated gastric fluid (without enzymes). Samples were collected at 0.5, 1, 2, 3, 4, 6, 8, 12 and 20 hours with replacement of equal volume of media. The samples were assayed at 280 nm using an HP diode array spectrophotometer for amoxicillin and at 219 nm for ranitidine HCl (Zantac®).

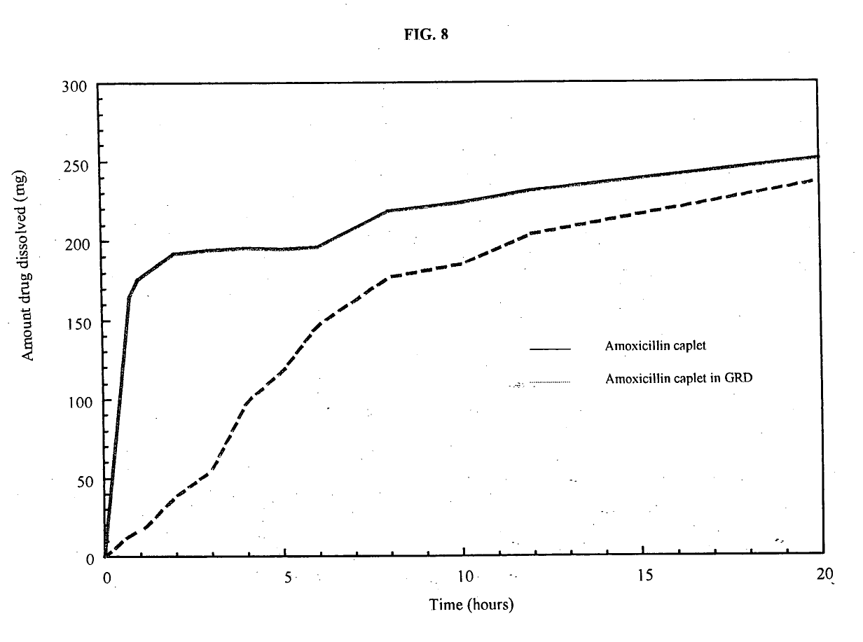

Dissolution studies of a) amoxicillin or b) ranitidine HCl tablets included in a GRD were compared with the formulations alone. Amoxicillin caplets were made as outlined in Example 6. The pattern of dissolution of amoxicillin immediate release (IR) tablet compared to the same formulation in a GRD is shown in FIG. 8. Amoxicillin IR released 80% drug in 1 hour; however only 10% drug release occurred from GRD at 1 hour, and 80% release was not reached until 12 hours. The release pattern of the drug from IR tablet incorporated into the GRD was zero-order.

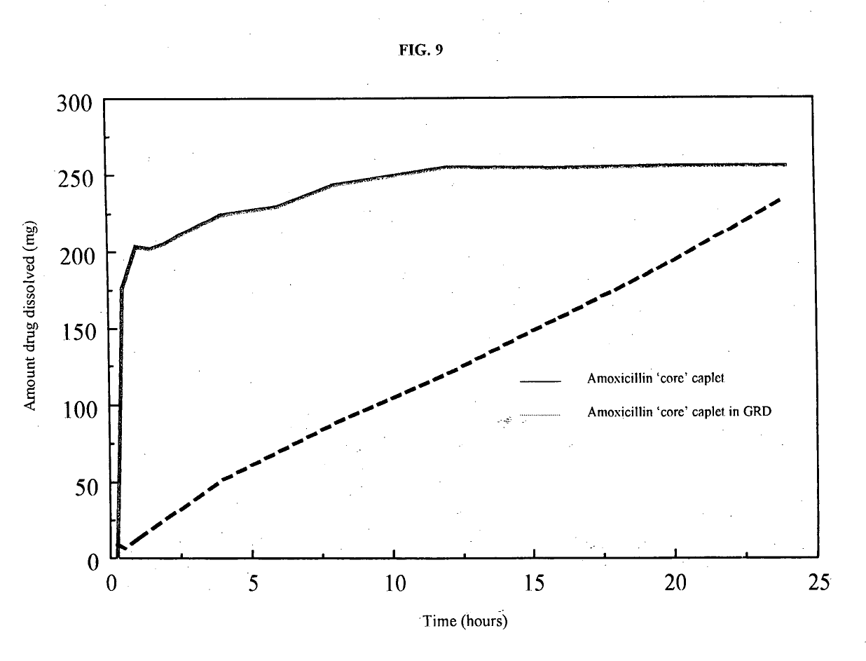

The dissolution of amoxicillin from a core tablet (amoxicillin caplet embedded in a microcrystalline cellulose shell) to that from a GRD containing the core tablet is presented in FIG. 9. Core tablet of amoxicillin released 80% drug in 1 hour, whereas the release of drug from core tablet inside a GRD was zero-order for 24 hours, and release of 80% of drug was over about 20 hours.

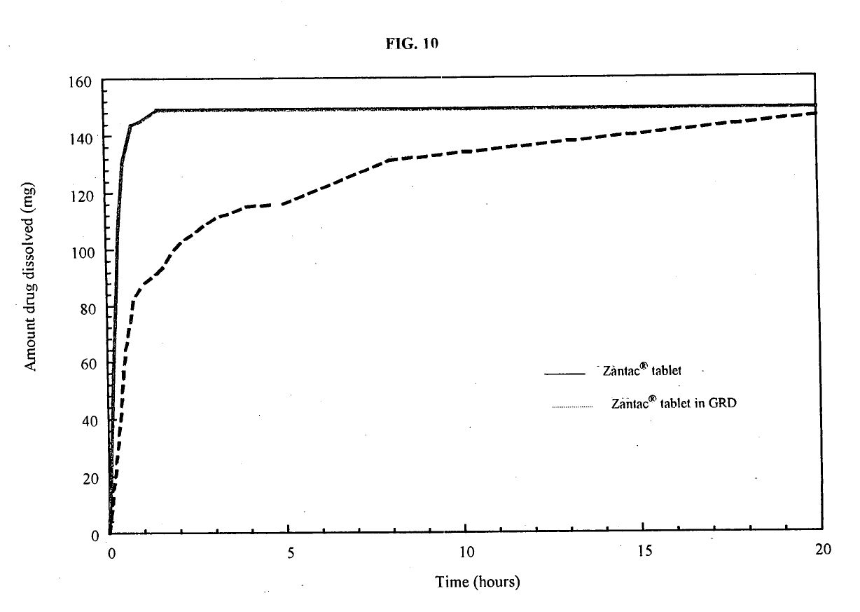

Comparison of dissolution from an immediate release, commercially available ranitidine HCl (Zantac® 150) tablet to that of an identical tablet incorporated into the GRD is presented in FIG. 10. Complete drug dissolution from the Zantac® 150 not in the GRD took 1 hour, where as only 80% drug release was observed in the first 7 hours from the tablet in a GRD.

B. Dissolution studies were carried out on GRDs prepared according to the methods of Example 1B that contained the model drug, riboflavin, using the USP XXII paddle method at 37° C. and 50 rpm for 24 hours. Dissolution medium consisted of 900 ml simulated gastric fluid without added enzymes. Samples were collected at 1, 2, 4, 6, 8, 10, 12, 16, 20, and 24 hours. The samples were assayed for riboflavin at 446 nm using a HP diode array spectrophotometer.

Dissolution studies of riboflavin beads, powder and solid dispersion included in GRD (regular or modified) were compared with immediate release capsule containing the same amount of vitamin. In all studies the amount of riboflavin was equivalent to 50 mg and the GRDs used were the rectangular shape (3*1.5*1). Size “0” capsules were used to fit both the immediate release formulation and the GRDs formulations.

Dissolutionfrom Regular GRD:

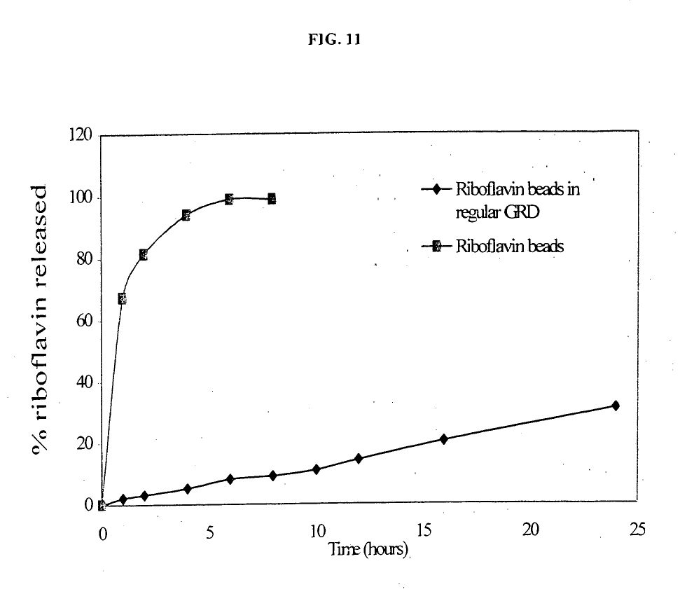

The pattern of dissolution of riboflavin beads contained in a capsule compared to the dissolution of riboflavin beads contained in the rectangular shape regular GRD is shown in FIG. 11. Riboflavin beads released 100% drug in 9 hrs, however only 8% drug release occurred from regular GRD at 5 hrs, and about 30% release at 24 hrs. The release pattern of drug from the regular GRD was nearly zero-order.

The dissolution of riboflavin powder from an immediate release capsule (50 mg riboflavin+200 mg lactose) was compared to that from a regular GRD containing the same amount of riboflavin powder. The immediate release capsule of riboflavin released 100% of drug in about 1 hr, whereas the GRD in a capsule released about 50% of drug in 24 hrs. The release of riboflavin powder from regular GRD was also nearly zero order.

Dissolution from Modified GRD:

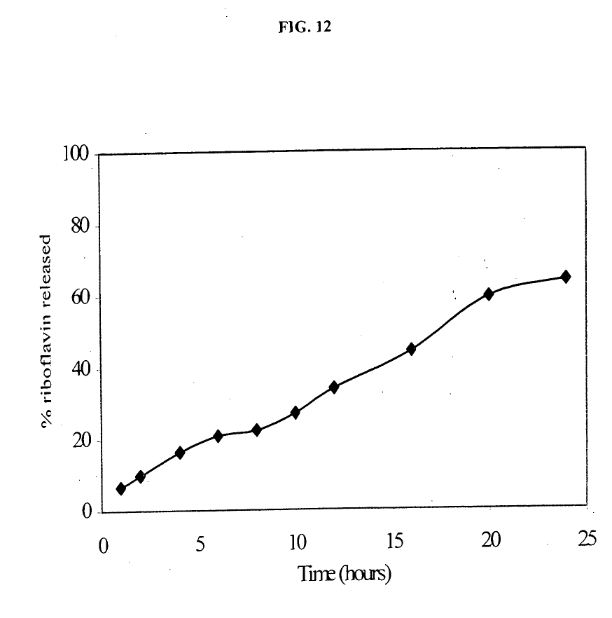

The prepared modified GRD was used to vary the rate and amount of drug release. The modified GRD differs from the regular GRD in that it contains PVP and SLS. The dissolution of riboflavin powder from the modified GRD is shown in FIG. 12. The modified GRD released about 65% of drug in 24 hrs. The pattern of release also looked zero order. The increased dissolution from the modified GRD may be attributed to the presence of the hydrophilic polymer PVP and the surface-active agent, SLS. Both PVP and SLS may have helped diffusion of the vitamin from the hydrogel. The presence of PVP and SLS in the formulation also produced more flexible dried films that were easier to fit into capsules when compared to the regular films from the formulation without PVP and SLS. The increased flexibility facilitates in fitting larger GRD in capsules.

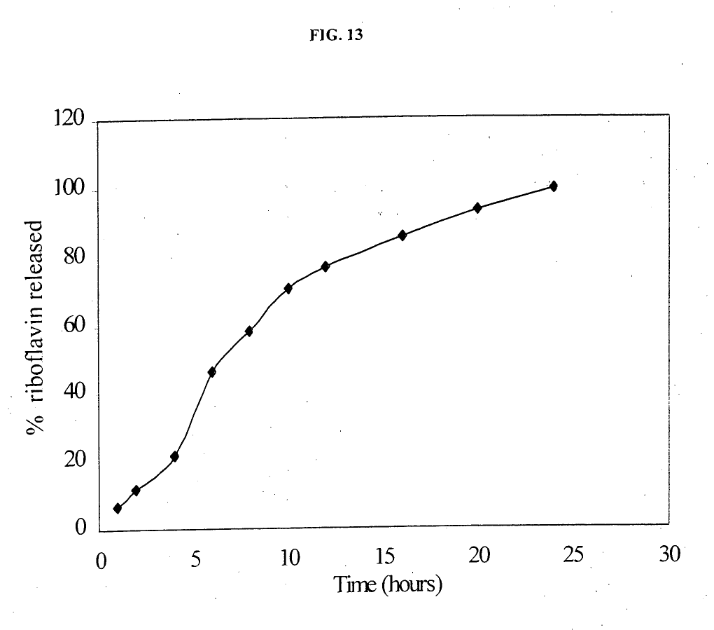

Dissolution of solid dispersion of riboflavin and PEG 3500 in the ratio 1:3 from modified GRD is shown in FIG. 13. It was observed that 100% of drug was released in 24 hrs from this formulation, however the GRD lost its integrity in about 6 hrs.

When the solid dispersion of riboflavin and PEG 3500 is added to the hot viscous gums' solution, soft films were produced when the gel was dried. After hydration for sometime in GF, this gel fell apart into fragments.

### Example 9

This section concerns subjects for in vivo testing of GRDs in dogs

A. Subjects for in vivo testing of GRDs made according to Example 1A, section IV

Two mixed-breed dogs aged 2.5 and 5 years were used to study the gastric residence time of different sizes and shapes GRDs. The animals were at the animal research lab in the Oregon State University College of Veterinary Medicine, and were maintained on a canned protein diet (d/d Hills) for two weeks. They were housed in individual pens that allowed reasonably free movement and normal activity of the dogs and thus normal gastrointestinal motility is expected.

B. Subjects for in vivo testing of GRDs made according to Example 1B

The studies were conducted in two adult German Shepherd dogs aged between 8 and 9 years. They were maintained on a commercially available feed and were at the animal research lab in the Oregon State University College of Veterinary Medicine. They were housed in small adjacent individual pens with rubberized wire mesh overlying concrete floors with a slope to facilitate sanitation. The animal pens allowed a reasonable space for free movement and normal activity of the dogs and thus there would be normal gastrointestinal motility. The housing area was kept lit during the daytime and dark at night.

### Example 10

This section concerns dosage forms and dosing of subjects for in vivo testing of GRDs in dogs

A. Dosage Forms for in vivo testing of GRDs made according to Example 1A, section IV

GRDs were administered to the subjects described in Example 9A. Four different shapes of GRDs incorporated in size “0” capsules were used. A 7×1.5×1 cm rectangular shape GRD incorporated in ‘000’ capsule also was tested in these studies. All the dosage forms contained radio-opaque threads for X-ray visualization.

Four different shaped GRDs incorporated into size ‘0’ capsules were tested in dogs to determine gastric residence time. The dimensions of these four shapes are listed in FIG. 8. All GRDs contained not less than 10 small pieces of radio-opaque threads. These threads helped visualize the GDRs in the GI tract by X-rays. They also helped in viewing the hydration and disintegration of the gels.

Dogs were fasted overnight. Dosage forms loaded with radio-opaque threads were administered orally early in the morning with 4 ounces of water.

Food was also mixed with BIPS and given 2 hours after dosing to study the effect of GRD on food emptying from the stomach. Two different sized GRDs were tested. One was incorporated in size ‘0’ capsule and the other in size ‘000’ capsule. These 2 sizes correspond to 3×1.5×1 and 7×1.5×1 cm respectively.

B. Dosage Forms for in vivo testing of GRDs made according to Example 1B

GRDs were administered to the subjects described in Example 9B. A gastric retention device enclosed in ‘000’ capsule containing barium sulphate caplets, radio-opaque threads, or bismuth impregnated polyethylene spheres (BIPS) was used. The system was followed using X-rays.

Dogs were fasted overnight and dosage forms were administered orally early in the morning with 10 ounces of water. Food was provided 3 hours after-dosing. A radiograph was taken just prior to dosing to ensure that the stomach was empty. The gastric retention device was followed by X-ray and the dogs were fed 3 hours after dosing. Presence of food can be readily recognized in X-rays as a darker area in the stomach.

Studies were carried out with the formulations containing different types of radio-opaque agents, such as barium sulphate tablets, radio-opaque threads and radio-opaque BIPS in the same dogs on different days. Normal gastric emptying of radio-opaque marker in the dogs under the conditions of fasting was determined by feeding a capsule containing radio-opaque threads.

BaSO₄ tablets were made in a Carver press in the shape of a caplet. Various methods were explored to incorporate the tablet. Basically, the method included pouring a layer of gel into a mold, putting tablets into the mold at desired distances, and immediately adding another gel layer. These gels were dried under vacuum. Dried films were compressed into a ‘000’ capsule. On subjecting these films to hydration studies, films were found to separate into two layers after hydration, and release the tablet prematurely.

The caplets were suspended with the help of threads in such a way that they stood in the middle of the inner side of the mold. When poured, hot gel entrapped the caplet. BaSO₄ was found to leak from the gel or tablet during gel expansion studies which would make it difficult to determine GRD location. Keeping this limitation in view, in vivo studies in dogs were carried out. As expected, it was difficult to trace the system in the stomach of dogs since the BaSO₄ tablet dissolved and spread throughout the GIT.

### Example 11

This Example concerns radiography for in vivo testing of GRDs in dogs.

A. Radiography for in vivo testing of GRDs made according to Example 1A, section IV

GRDs were administered as described in Example 10A. Radiographic examinations were performed using a Transworld 360 V X-ray generating unit. X-ray cassettes used were 3 M Trimax 12 paired with 3M ultradetail (1416) film. Radiography was used to follow passage of GRDs in the GI tract. Radiographs for dogs were exposed at 0 minutes Oust before dosing to ensure an empty stomach), at 5 min Oust after dosing to assure that the device is in the stomach), at 2 hours (to see if the GRD is not removed by the housekeeper wave), and at 9 hours. The dogs were fed after the 2 hours radiographs. Food was sometimes mixed with BIPS (barium impregnated polyethylene spheres) to study the effect of the dosage form on food emptying from the stomach. BIPS have a density similar to food but are sufficiently radiodense to show clearly on abdominal radiographs. The small BIPS used (1.5 mm) mimic the passage of food and their transit through the GI tract provides an accurate estimate of the gastric emptying rate and intestinal transit time of food. Hills d/d diet is known to suspend BIPS and it is the only diet in which the correlation between BIPS emptying and food emptying has been investigated and proven. BIPS can be differentiated from radio-opaque threads in radiographs. For each animal, radiographic examinations were performed from two angles, a lateral view and a dorsoventral view.

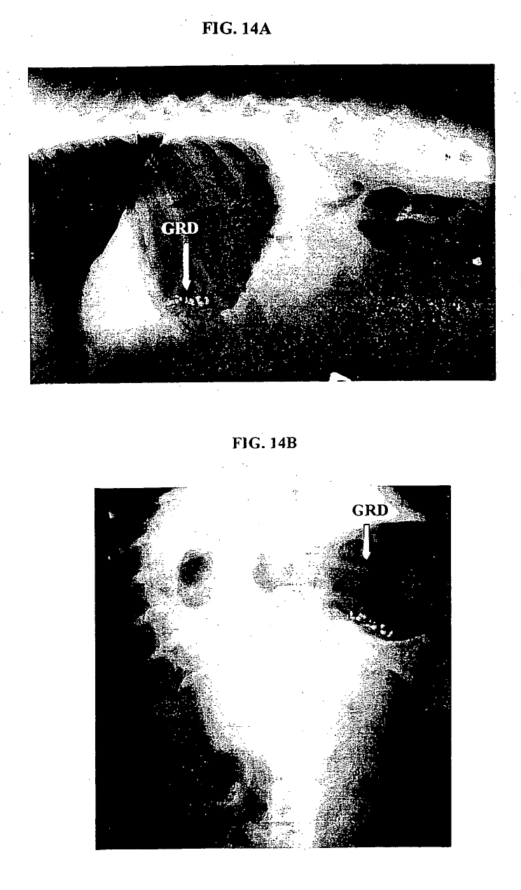

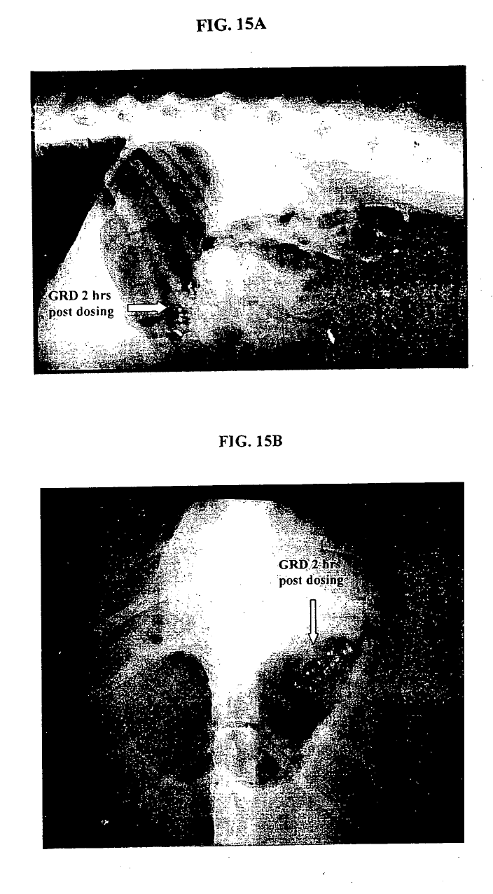

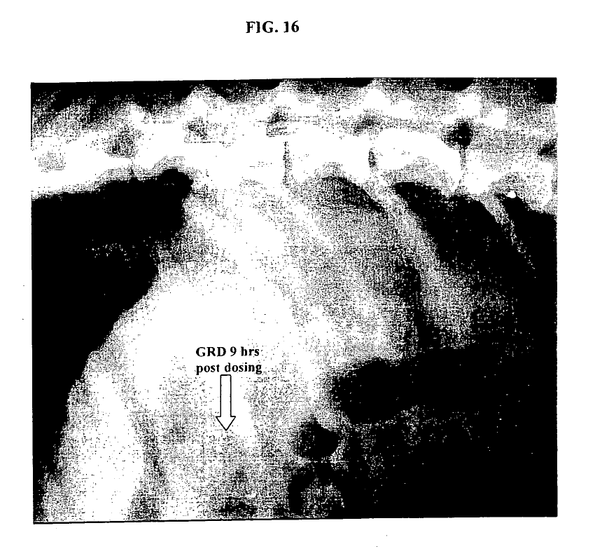

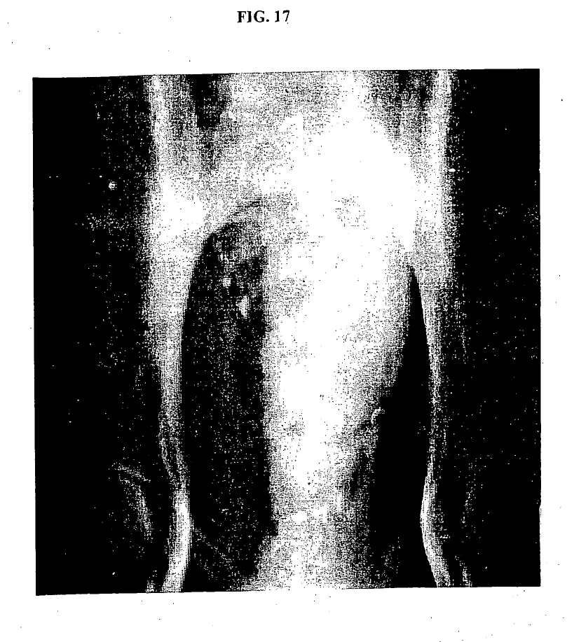

The rectangular shape was found to stay in the stomach of one of the dogs for at least 9 hours. The other three shapes were emptied from the stomach in less than 2 hours. X-rays at 24 hours indicated absence of radio-opaque threads in the stomach for the rectangular shape GRD, and disintegration of the four different shape GRDs as indicated by the spread of threads in the colon. A total of four studies were conducted using the rectangular shape GRD. In all four studies the GRDs stayed in the stomach of the same dog but not in the other one. The results of these studies are shown in FIGS. 14-17.

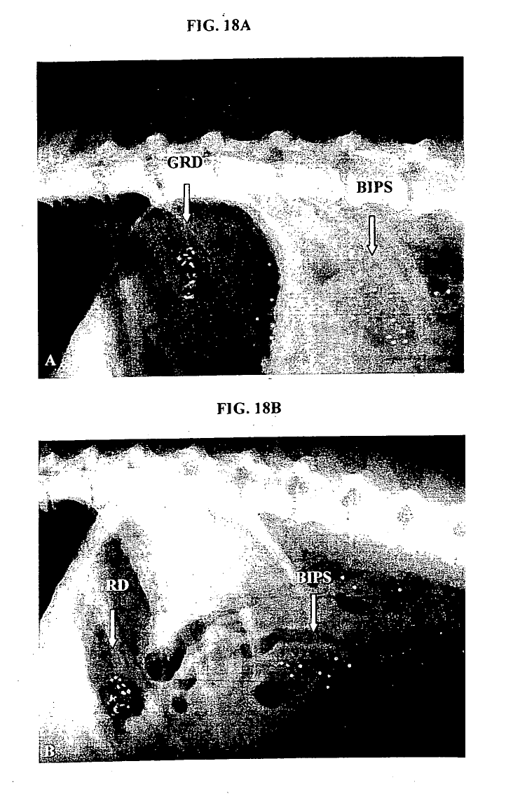

X-rays taken 2 hrs after food mixed with BIPS showed the food has emptied from the stomach while GRDs did not. The results of this study are depicted in FIG. 18. This indicates that GRD did not affect food emptying from the stomach into the intestine. The result from the larger size GRD also indicates that the pyloric sphincter was not blocked by GRD. Based on the results from this in-vivo study, the large size GRD incorporated in ‘000’ capsule was chosen to test in humans.

B. Radiographyfor in vivo testing of GRDs made according to Example 1B

GRDs were administered as described in Example I OB. X-rays were employed to follow the passage of the gastric retention device in gastrointestinal tract of dogs. Radiographs were taken just before dosing to ensure an empty stomach and immediately after dosing. Subsequent X-rays were taken at 0.5 hour, 1 hour, 2, 3, 6, 9, and 24 hours. All X-rays were lateral view, and some anterioposterior (ventrodorsal, VD) X-rays were also taken to confirm the position of the dosage form in the dog stomach.

Radiographic examinations were performed using a Transworld 360V X-ray generating unit (360 milliamperage and 125 kilovoltage potential). X-ray cassettes used were 3M Trimax 12 paired with 3M Ultradetail (1416) film. Exposure settings are shown in Table 5.

TABLE 5
Exposure settings of X-ray machine for the two dogs.

| Dog | mA | KVP | MAs |
|---|---|---|---|
| Hans-lateral view | 150 | 70 | 8.3 |
| Gretel-lateral view | 150 | 68 | |
| Hans-VD view | 150 | 82 | 10.1 |
| Gretel-VD view | 150 | 80 | |

Bismuth Impregnated Polyethylene Spheres (BIPS), as the name implies, are polyethylene spheres containing bismuth and this makes them radio-opaque. These spheres were incorporated in the GRD for study in dogs. The system containing two large BIPS was followed with X-rays at different time points including 0, 0.5 hr, 1 hr, 2, 3, 6, 8, 9, and 24 hours. The system was present in the stomach of one of the dogs at the 9th hour of experimentation. The next X-ray was not taken until 24 hours. Of the 2 BIPS, one was still in the stomach, whereas the other one was found in the intestine, indicating that the system must have eroded with the release of one BIPS. In the case of the second dog, both BIPS were found in the small intestine at 9 hours.

Radio-opaque threads have been used in veterinary medicine and surgery, and pieces of these threads were incorporated in the GRD. These threads help not only in tracing the film but also in viewing gel hydration.

A placebo study was carried out in both the dogs. Capsules with radio-opaque threads and lactose were administered to dogs under the conditions of fasting to study the aspects of gastric emptying of the threads when not in a GRD. X-rays were taken at regular intervals. These threads were eliminated from the stomach of dogs into the small intestine between 2 and 3 hours.

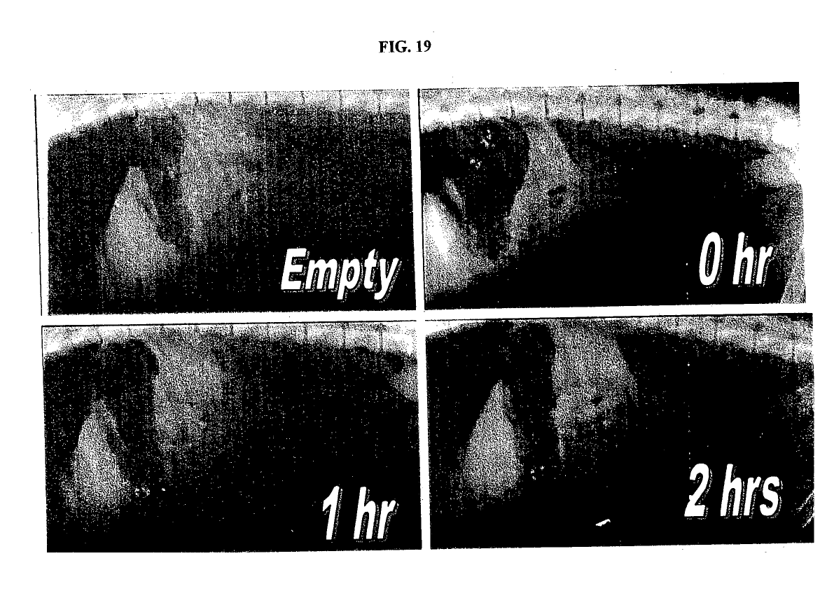

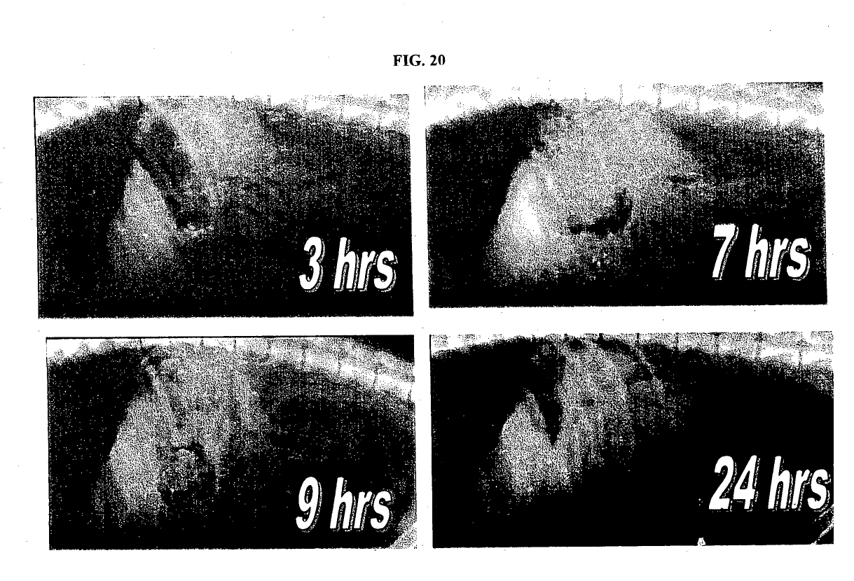

The administration of a gastric retention device containing radio-opaque threads to dogs was also followed with X-rays. The system stayed in the stomach of dogs for at least 10 hours. The X-rays taken at 24 hours demonstrated absence of radio-opaque threads either in the stomach or in the small intestine. The results of administration of GRD-containing, radio-opaque threads in dogs are presented in FIGS. 19 and 20. A total of 5 studies were conducted using GRDs containing radio-opaque materials. The system was found to stay in the stomach of dogs for at least 9 hours, as observed in 3 of our studies.

X-rays taken at or after 7 hours of dosing showed absence of food in the stomach and food was found in the intestine. However the GRD was found in the stomach. This indicates that GRD did not affect the passage of food into intestine and did not block the pyloric sphincter by GRD.

### Example 12

This section concerns endoscopy for in vivo testing of GRDs in dogs

Endoscopy was used to allow visual observation of swelling in the stomach of GRDs made according to Example 1A, part IV. One dog was used for this study. The animal was fasted 14-16 hr prior to dosing. The dog was dosed while awake. The animal was induced with ketamine (259 mg) in combination with diazepam (7.5 mg) given intravenously. The animal was intubated with a cuffed endotracheal tube and maintained under general anesthesia with isoflurane gas and oxygen. Following attainment of a suitable anesthetic plane, a flexible fiber optic endoscope (135 cm length; 9 mm o.d.) was introduced into the mouth and esophagus and guided to the stomach. The GRD was monitored by a camera attached to the endoscope, and the expansion process was recorded on videotape over a period of 45 minutes.

The animal was scheduled for endoscopic exam, and the endoscopic procedure was well tolerated. The total procedure time, as defined as the time from anesthetic induction to extubation, was about 1 hr. The endoscope was directed to the stomach of the animal. Endoscopic views showed the location of GRD in the stomach. The GRD was then monitored continuously by the endoscopic camera over a period of 45 minutes. The capsule shell dissolved in few minutes and the GRD was released. The GRD swelling occurred gradually over a period of 30 minutes. After 45 minutes the swollen gel was recovered from the stomach to study its dimensions and compare it to in vitro results. The recovered swollen gel from the dog stomach reached about the same dimensions (2.8*1.3*0.8) as compared to a similar GRD immersed in simulated gastric fluid at 37° C. (3*1.5.1). The prepared GRD swells to a considerable size in gastric fluid in less than 30 minutes and therefore has a good chance to avoid removal from a fasted stomach by the housekeeper wave.

### Example 13

This section concerns administration of GRDs to humans.

A. Administration of GRDs to human subjects using GRDs made according to the method of Example 1A, section IV

A cross over bio-study under fasted and fed conditions was conducted in one subject for a gastric retention device containing 200 mg of amoxicillin or just the 200 mg amoxicillin tablet without the device. The subject was asked to fast overnight in both studies. During the study, under conditions of fasting, breakfast was provided two hours after dosing. In the fed state study, the subject received the dosage form with breakfast. The standard breakfast was a plain bagel, one ounce of cream cheese and 125 ml of fruit juice. After a washout period of 48 hours, the alternate dose was given. Urine was collected at 0 hr, 1 hr, 2, 3, 4, 5, 6, 8, 12 and 24 hours. Urine samples were analyzed immediately by HPLC.

B. Administration of GRDs to human subjects using GRDs made according to the method of Example 1B

Phase I.

Six healthy subjects (four males and two females) ingested either an (IR) capsule (Treatment A) or (LGRD) capsule (Treatment B) in a randomized crossover design with a washout period of at least one week. The capsules were ingested with 200 ml of water. All subjects were asked to fast for at least 10 hours before the study and no food was allowed for three hours after dosing.

Phase II.

This study consisted of one treatment under fasting conditions, where each of the six subjects ingested an (IGRD) capsule (Treatment C).

Phase III:

This study consisted also of one treatment under fasting conditions, where each of the six subjects ingested a (SGRD) capsule (Treatment D).

Formulation Ingredients

Riboflavin was selected as the therapeutic (Sigma Chemicals, St. Louis, Mo.). All test formulations, either in form of GRD or immediate release containing 100 mg riboflavin in powder form, were produced at College of Pharmacy, Oregon State University, Corvallis, Oreg. GRD formulations were prepared as described previously.

Capsules used in the Biostudy

1. Immediate release (IR) capsules: were size “1” capsules that contained lactose as the principal excipient (200 mg) and 100 mg of previously dried riboflavin.

2. Large GRD capsules (LGRD): were size ‘000’ capsules filled with dried GRD containing 100 mg riboflavin. The dimensions of incorporated GRD before drying were 7*1.5*1 cm.

3. Intermediate GRD capsules (IGRD): were size ‘00’ capsules filled with dried GRD containing 100 mg riboflavin. The dimensions of the incorporated GRD before drying were 5*1.5*1 cm.

Small GRD capsules (SGRD) were size ‘0’ capsules filled with dried GRD containing 100 mg riboflavin. The dimensions of the incorporated GRD before drying were 3* 1.5*1 cm.

### Example 14

This section concerns HPLC analysis of drug excretion following administration of GRDs to human subjects.

A. HPLC analysis of urine samples from the subject of Example 13A.

Internal Standard: Acetaminophen USP (1 mcg/ml).

This solution is relatively stable when stored cold and well protected from direct light.

Buffer solution:

The buffer was prepared by adding 100 ml 0.5M disodium hydrogen phosphate to 350 ml deionized water. The pH is adjusted to 6 with 1M citric acid. The resulting solution is made up to 500 ml volume with deionized water. Mobile phase preparation: 0.26 g potassium dihydrogen phosphate was added to 3800 ml of deionized water. 200 ml HPLC grade methanol was added. The solution was filtered to remove any particulate and stirred under vacuum for approximately 20 minutes to remove air bubbles.

HPLC instrument: Waters Intelligent Sample Processor (WISPTM) 712, automatic sample injection module for up to 48 sample vials for injection on to the column.

Column: Reverse phase C18, 25 cm, 5 micron, 100A Rainin Microsorb-MV®

Detector: UV absorbance detector, Model 440 with fixed wavelength.

Buffered sample: 2 ml from each urine sample are added to 2 ml pH 6 buffer.

The solution is vortex-mixed to ensure proper mixing.

HPLC sample: 1 ml buffered urine was diluted with 5 ml deionized water. To 50 microliters of this diluted sample, 50 microliters internal standard solution was added in a small plastic centrifuge tube. The resulting solution was vortex-mixed to ensure mixing. The HPLC sample vial was assembled and capped and placed in a WISP™ autoinjector for HPLC analysis. 20 microliters of sample was injected. All other parameters for HPLC are listed below.

Flow rate of mobile phase: 1.3 ml/minute

Wavelength of detection: 229 nm

Run time: approx. 23 minutes.

Generation of a Standard Curve

An amoxicillin calibration curve was generated by the following method: 0.03 g amoxicillin trihydrate was placed in a 100 ml volumetric flask, dissolved and made up to 100 ml with 1:10 mixture of drug-free (blank) urine: deionized water. This was stirred at room temperature for approximately 40 minutes to ensure complete dissolution. A series of 1:1 dilutions are made with deionized water to obtain 6 samples. This process of serial dilution resulted in a series of samples within a range of concentrations that was used to produce the calibration curve. The method of sample preparation for HPLC analysis was as given previously. A total of 20 microliters of each sample was injected.

B. HPLC analysis of urine samples from the subjects of 13B

1) Reagents for HPLC Assay:

Methanol (HPLC grade, Fisher Chemicals, N.J.), Potassium monobasic phosphate (Fisher Chemicals, N.J.), Sodium hydroxide (Mallinckrodt). The water used in this procedure was deionized using the Milli-Q Reagent Water System (Millipore, Bedford, Mass., USA).

2) Drug Assay Method:

The column was a reversed-phase micro-particulate C₁₈ (□ Bondapak C₁₈, particle size 10 μm, 30 cm×4 mm, Waters Assoc., Milford, Mass., USA.) preceded by a C₁₈ guard cartridge (ODS, 4×3 mm, Phenomenax, Calif., USA).

Assay procedure followed that described by Smith. The eluent was 0.01 m KH₂PO₄ (pH 5): methanol (65:35) at a flow rate of 1.2 ml/min. The mobile phase was prepared by mixing exact volumes of methanol and 0.01 potassium monobasic phosphate solution adjusted to pH 5 with 1 N sodium hydroxide and then filtering under vacuum through a 0.2 μm filter. The mobile phase was degassed before use. The detector was a fixed-wavelength spectrofluorometer (Gilson Spectra/Glo Fluorometer, Middleton, Wis.). The excitation wavelength was 450 rm. The wavelength range for the emission filter was 520-650 nm. Peak areas were determined with a Schimadzu integrator (C-R3A Chromatopac, Schimadzu Corp., Kuoto, Japan).

Other instruments in the HPLC system included a delivery pump (Waters 550 Solvent Delivery System, Waters Associates, Milford, Mass.), an automatic sample injector (Waters WISP Model 712B, Waters Associates, Milford, Mass.).

3) Collection of Urine Samples:

Subjects fasted overnight, provided a zero-time urine sample prior to dosing, then ingested a formulation. Urine samples were collected in 16 oz containers at 1, 2, 3, 4, 6, 8, 10, 12, and 24 hours post dosing. Volume and time elapsed since vitamin ingestion were recorded for each urine sample and a portion was saved for vitamin concentration measurement.

4) Standard Solutions:

Riboflavin standard stock solutions were prepared to contain 100 μg/ml of reference standard by addition of 100 mg of riboflavin, previously dried at 105° C. for 2 hours, 750 ml of water and 1.2 ml of glacial acetic acid to a 1-liter flask, dissolving with heat, and diluting to volume with water. This stock solution was diluted with blank urine to contain 1, 2, 4, 6, 8, 10, and 15 μg/ml of riboflavin. All solutions were protected from light. These standards were injected onto the column, the chromatogram was recorded and the peak areas determined. The retention time of riboflavin was about 6 minutes.

A standard curve was constructed by plotting peak areas against riboflavin concentration in urine. Assay sensitivity was 1 μg/ml with linear relationship between peak areas and riboflavin concentrations of 1 to 10 μg/ml (R²=0.9971). A typical standard curve for riboflavin in urine is shown in FIG. 29. Endogenous riboflavin was taken into account by subtracting the area obtained from the analysis of blank urine or zero time urine sample from all assayed standards and samples.

5) Sample Analysis:

Approximately 10 ml of urine were centrifuged at 4000 rpm for 10 minutes. A portion of the supernatant (150 μl) was transferred to HPLC tubes and 50 μl was injected onto the HPLC column. Riboflavin eluted 6 minutes after injection.

### Example 15

This section concerns pharmacokinetics analysis of HPLC data.

Riboflavin excretion data was obtained as outlined in Example 14B, sections 1-5. The different treatments were compared in terms of their urinary recovery of riboflavin during the first 24 h after administration, Recovery₀₋₂₄, the maximum urinary excretion rate, Rₘₐₓ and the time, Tₘₐₓ required to reach Rₘₐₓ, All parameters were determined from the individual urinary excretion rate-time curves, a plot of urinary excretion rate against the mid-point of a urine collection interval. Recovery₀₋₂₄ₕ was determined from the individual cumulative urinary drug excretion-time curve, a plot relating the cumulative drug excreted to the collection time interval.

### Example 16

This section concerns statistical analysis of HPLC data.

Riboflavin excretion data was obtained as outlined in Example 14B, sections 1-5. Between-treatments differences in pharmacokinetic parameters were examined using a two-sided student t test. The two-sided student t test at α=0.05 on the null hypothesis Ho: μ_{τ}−μ_{R}=0 were performed on Recovery₀₋₂₄ₕ, Rₘₐₓ and Tₘₐₓ for urinary recovery data. Acceptance of the null hypothesis (Ho) indicates that there is not enough evidence to conclude a significant difference exists between the parameter mean of the GRD formulation and the corresponding parameter mean of the immediate release formulation; i.e. the parameters are equivalent. Rejection of the null hypothesis is a strong indication that the tested parameters of the two formulations are significantly different.

### Example 17

This section concerns deconvolution of urinary excretion rate data.

Deconvolved input finctions from biostudy data were determined using computer software PCDCON by Williams Gillespie. Deconvolution generates an input function (cumulative amount dissolved in vivo versus time) from an input response and the drugs' characteristic impulse response function. The cumulative drug input over time predicted by deconvolution was used to determine the gastric retention time of GRDs of different sizes. The gastric retention time was calculated from the deconvolved curve as the time observed when absorption stops. The input response used was the urinary excretion rate of riboflavin from the different formulations (dU/dt), while the impulse response used was a literature-derived elimination rate constant as determined from an intravenous bolus dose of riboflavin.

### Example 18

This section concerns drug absorption by human subjects from GRDs.

A. Amoxicillin excretion following administration of GRDs to a subject as outlined in Example 13A and analysis as outlined in Example 14A.

Amoxicillin (a β-lactam antibiotic) incorporated in a GRD in the form of a caplet was tested for its bioavailability. Elevation of β-lactam concentration demonstrates increased bacterial killing, only until a finite point that tends to be about 4 times the minimum inhibitory concentration (MIC), which can be termed as therapeutic concentration. Further elevation is not associated with increased bactericidal potency (18, e.g., MIC for Strep. pneumococci is 0.02 mcg/ml and therapeutic concentration is 0.08 mcg/ml). A direct correlation exists between the time the β-lactam antibiotic concentrations are maintained above therapeutic concentration and clinical actions. Bacterial regrowth occurs rapidly after these concentrations fall below the bacterial MIC. Therefore a dosage regimen for each individual β-lactam should be to prevent the drug-free interval between doses from being large enough for bacterial pathogens to resume growth.

Amoxicillin has a very short half-life of about 1 hour and a limited ‘absorption window’ following oral administration. Drug is well absorbed in duodenum and jejunum, but absorption is decreased in ileum and is rate dependent. Absorption is very poor in all colonic regions. Therefore, using GRDs to deliver β-lactam antibiotics such as amoxicillin would expand the time over MIC in vivo in relation to regular IR formulations. Bioavailability would also improve as amount of drug reaching the site of absorption is prolonged over a period of time and thus preventing saturation at that site.

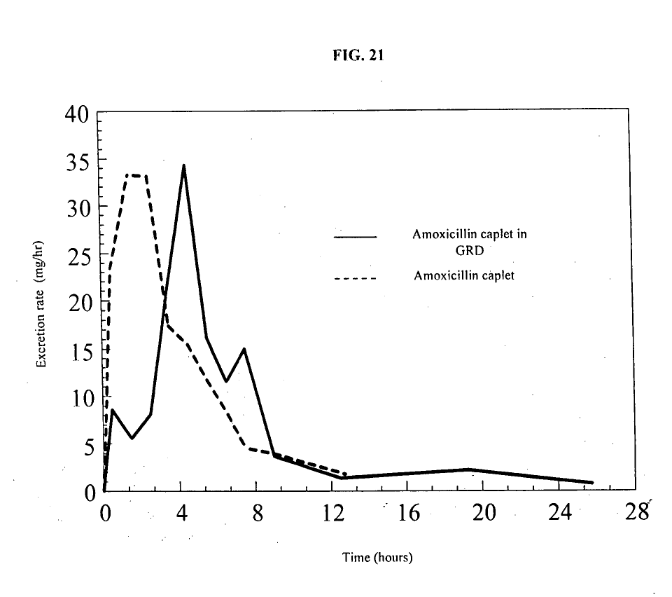

When amoxicillin was administered to the subject of Example 13A under fasting conditions, and analyzed according to the method of Example 14A, a 30% increase in area under the excretion rate curve (AUC) for drug incorporated into the GRD was found when compared with absence of GRD. The maximum excretion rates (Cₘₐₓ) were 34.2 mg/hr in absence of GRD and 29.0 mg/hr in presence of GRD and these values were not significantly different. The values of Tₘₐₓ were identical for both. Comparative bioavailabilities of the two formulations are illustrated in FIG. 21.

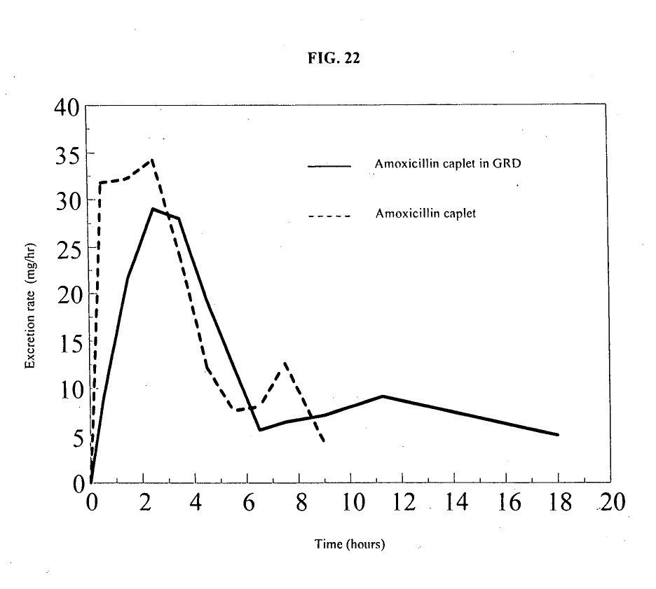

The study carried out under fed conditions did not show any significant difference in AUC or Cₘₐₓ. However Tₘₐₓ for GRD was shifted to the right compared to that in absence of GRD. The Tₘₐₓ for GRD was found to be 4 hours, where as, it was 2 hours in absence of GRD. The bioavailabities for both the formulations under fed conditions are given in FIG. 22.

These results with amoxicillin are consistent with food slowing drug delivery from the stomach to the intestine when a subject is fed, and the GRD slowing the delivery of drug to the intestine when the subject is fasted. Further, the food did not adversely influence drug release from the GRD.

B. Riboflavin excretion following administration of GRDs to subjects as outlined in Example 13B and analysis as outlined in Example 14B, sections 1-5 and Examples 15, 16,and 17.

Urinary drug excretion data can be used to estimate bioavailability because the cumulative amount of drug excreted in the urine is directly related to the total amount of drug absorbed and then excreted through a first-order elimination process. In order to obtain valid estimates, the drug must be excreted in significant amounts in the urine and complete samples of urine must be collected.

Determination of the cut-off size for gastric emptying of GRD under fasting conditions was one goal of this biostudy. Relative fractional absorption of riboflavin from the different formulations was evaluated from urinary excretion data. Mean pharmacokinetic parameters for the different treatments are shown in the following table.

TABLE 6
Pharmacokinetic parameters of riboflavin after oral
administration of 100 mg in immediate release or GRD
capsules to fasted volunteers.
Treatments

| | (IR) | (SGRD) | (IGRD) | (LGRD) |
|---|---|---|---|---|
| Recovery | 5.33 ± 1.74 | 4.09 ± 1.67 | 9.3 ± 5.27 | 17.36 ± 9.7 |
| from | | | | |
| 0-24 h (mg) | | | | |
| Max. | 1.36 ± 0.42 | 1.14 ± 0.59 | 2.05 ± 0.99 | 2.52 ± 0.98 |
| Urinary | | | | |
| excretion | | | | |
| rate (mg/h) | | | | |
| Time of | 2.5 ± 0.6 | 2.33 ± 0.97 | 3.25 ± 1.1 | 5.08 ± 2.4 |
| max. | | | | |
| excretion | | | | |
| rate (h) | | | | |
| Mean | 4.73 ± 0.83 | 5.98 ± 1.06 | 5.27 ± 1.7 | 6.99 ± 1.18 |
| Residence | | | | |
| time (h) | | | | |

Data are mean values ± SE

Individual pharmacokinetic parameters for each subject for the four treatments are also shown in Tables 7-12 below.

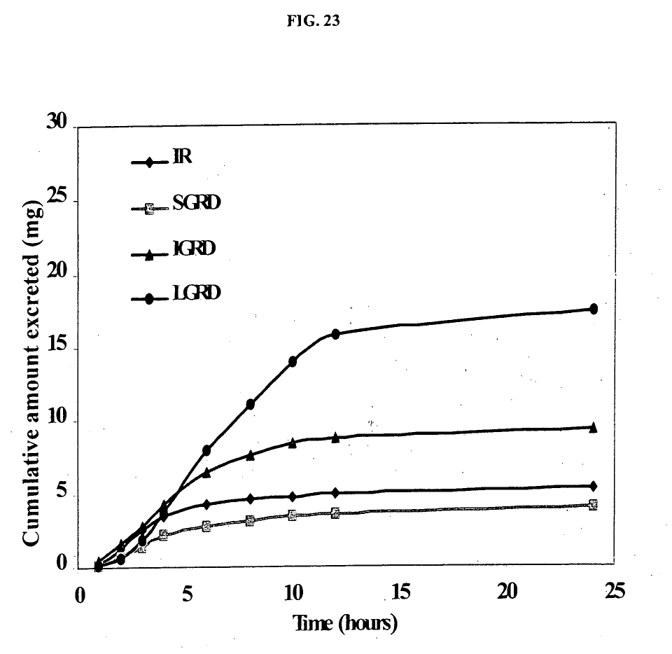

FIG. 23 shows that the largest mean value for Recovery₀₋₂₄ₕ was observed for LGRD capsule, followed by IGRD capsule, IR capsule, and SGRD capsule. The mean Recovery₀₋₂₄ₕ estimate from the LGRD capsule (17.3 mg) was determined to be 225% larger and statistically significantly (P<0.05) different relative to the mean from IR capsule (5.33 mg). Mean Recovery₀₋₂₄ₕ estimate from SGRD capsule (4.09 mg) was less but not statistically significantly (P<0.05) different relative to the mean from the IR capsule (5.33 mg). The mean Recovery₀₋₂₄ₕ estimate from the IGRD capsule (9.3 mg) was higher but not significantly different from the IR capsule. This could be due to prolonged gastric residence time of the device in only some of the volunteers (subjects 1 and 2 had significantly higher urinary Recovery₀₋₂₄ₕ from IGRD capsule when compared to the IR capsule).

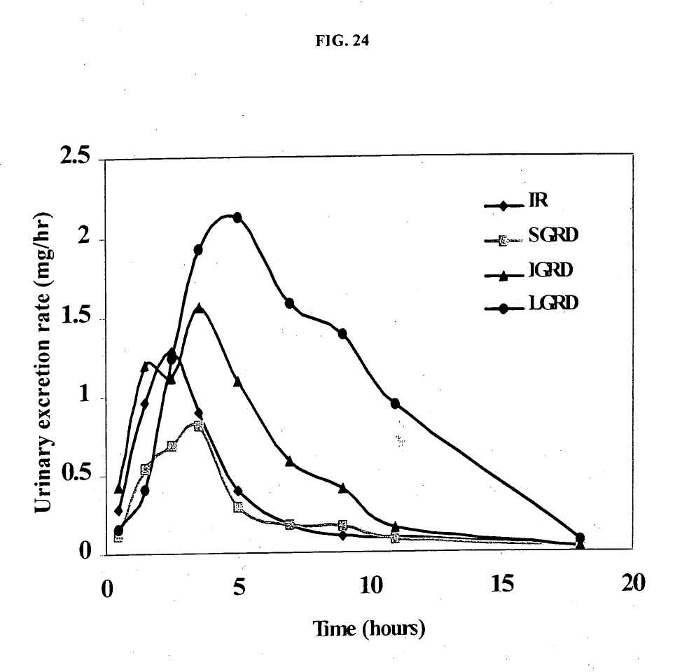

Statistical comparison of Rₘₐₓ and Tₘₐₓ parameters also indicated a significant difference (P<0.05) between results from LGRD capsule (2.5±0.98 mg/h and 5.08±2.4 hr respectively) and the IR capsule (1.36±0.4 mg/h and 2.5±0.63 hr respectively). Rₘₐₓ and Tₘₐₓ parameters from IGRD and SGRD capsules were not significantly different from the (IR) capsule. These results are shown in FIG. 24.

The improved bioavailability of riboflavin from the LGRD capsule (urinary recovery was more than triple that measured after administration of the IR capsule) obtained in this study, suggests that the device was retained in the stomach. The LGRD stayed in the stomach for enough time to slowly release its vitamin content and consequently the released vitamin passed gradually through the absorption window and was absorbed more efficiently.

Administration of the SGRD capsule, on the other hand, resulted in reduction of riboflavin absorption when compared with the IR capsule. This could be due to the small size of the device that was emptied from the stomach by phase III myoelectric migrating contraction activity with relatively little drug released. Once the device passes the absorption window, no absorption takes place.

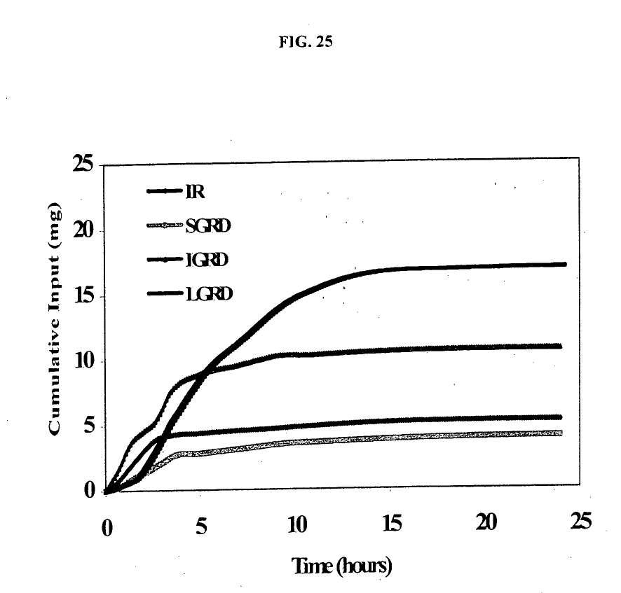

FIG. 25 shows the cumulative amount of drug absorbed versus time deconvolved from biostudy data for the IR, SGRD, IGRD, and LGRD capsules. Absorption continued for up to 15 hours for the LGRD capsule before it stopped. This may indicate that the LGRD stayed in the stomach and slowly released the drug for about 15 hours. The absorption from the IGRD capsule, on the other hand, continued for about 9 hours before it became constant, indicating that the device did not stay long enough in the stomach to release all of its' drug content. Absorption from SGRD capsule continued only for 3 hours indicating that the device was emptied from the stomach by the housekeeper wave (due to its small size) as rapidly as the IR dose.

These results indicate that gastric residence time of swellable systems such as GRD containing different drugs with limited absorption sites can be evaluated by comparing drug bioavailability, as determined by measurement of AUC or urinary recovery, after administration of the swellable system and an immediate release system containing the same amount of drug.

TABLE 7
Pharmacokinetic parameters of riboflavin after oral
administration of 100 mg in immediate release or GRD
capsules to subject 1
Treatments

| | (IR) | (SGRD) | (IGRD) | (LGRD) |
|---|---|---|---|---|
| Recovery from 0-24 h (mg) | 8.001 | 6.0 | 17.92 | 33.75 |
| Maximum Urinary excretion rate | 1.93 | 1.94 | 2.27 | 4.06 |
| (mg/h) | | | | |
| Time of maximum urinary excretion | 2.5 | 3.5 | 5 | 9 |
| rate (h) | | | | |
| Mean Residence time (h) | 4.08 | 5.421 | 6.49 | 7.59 |

TABLE 8
Pharmacokinetic parameters of riboflavin after oral
administration of 100 mg in immediate release or GRD
capsules to subject 2:
Treatments

| | (IR) | (SGRD) | (IGRD) | (LGRD) |
|---|---|---|---|---|
| Recovery from 0-24 h (mg) | 4.67 | 5.57 | 12.87 | 23.89 |
| Maximum Urinary excretion rate | 1.44 | 0.95 | 2.82 | 2.05 |
| (mg/h) | | | | |
| Time of maximum urinary excretion | 1.5 | 2.5 | 5 | 11 |
| rate (h) | | | | |
| Mean Residence time (h) | 3.40 | 7.52 | 5.90 | 8.70 |

TABLE 9
Pharmacokinetic parameters of riboflavin after oral
administration of 100 mg in immediate release or GRD
capsules to subject 3:
Treatments

| | (IR) | (SGRD) | (IGRD) | (LGRD) |
|---|---|---|---|---|
| Recovery from 0-24 h (mg) | 6.3 | 3.89 | 6.86 | 14.12 |
| Maximum Urinary excretion rate | 1.26 | 1.62 | 2.64 | 2.46 |
| (mg/h) | | | | |
| Time of maximum urinary excretion | 2.5 | 1.5 | 1.5 | 3.5 |
| rate (h) | | | | |
| Mean Residence time (h) | 5.61 | 4.38 | 3.17 | 5.73 |

TABLE 10
Pharmacokinetic parameters of riboflavin after oral
administration of 100 mg in immediate release or GRD
capsules to subject 4:
Treatments

| | (IR) | (SGRD) | (IGRD) | (LGRD) |
|---|---|---|---|---|
| Recovery from 0-24 h (mg) | 3.47 | 4.34 | 6.51 | 12.50 |
| Maximum Urinary excretion rate | 0.91 | 1.34 | 1.52 | 1.55 |
| (mg/h) | | | | |
| Time of maximum urinary excretion | 3.5 | 3.5 | 3.5 | 7 |
| rate (h) | | | | |
| Mean Residence time (h) | 4.91 | 6.13 | 5.01 | 7.13 |

TABLE 11
Pharmacokinetic parameters of riboflavin after oral
administration of 100 mg in immediate release or GRD
capsules to subject 5:
Treatments

| | (IR) | (SGRD) | (IGRD) | (LGRD) |
|---|---|---|---|---|
| Recovery from 0-24 h (mg) | 3.63 | 1.30 | 3.11 | 6.46 |
| Maximum Urinary excretion rate | 0.907 | 0.43 | 0.42 | 1.4 |
| (mg/h) | | | | |
| Time of maximum urinary excretion | 2.5 | 1.5 | 2.5 | 2.5 |
| rate (h) | | | | |
| Mean Residence time (h) | 5.33 | 5.86 | 7.55 | 5.55 |

TABLE 12
Pharmacokinetic parameters of riboflavin after oral
administration of 100 mg in immediate release or GRD
capsules to subject 6:
Treatments

| | (IR) | (SGRD) | (IGRD) | (LGRD) |
|---|---|---|---|---|
| Recovery from 0-24 h (mg) | 5.96 | 3.45 | 8.81 | 13.53 |
| Maximum Urinary excretion rate | 1.77 | 0.58 | 1.88 | 3.06 |
| (mg/h) | | | | |
| Time of maximum urinary excretion | 2.5 | 1.5 | 3.5 | 5 |
| rate (h) | | | | |
| Mean Residence time (h) | 5.06 | 6.62 | 3.53 | 7.28 |

### Example 19

This section concerns Production of a Gastric Retention Device containing hydrochlorothiazide

All ingredients and molds were prepared (a 1*1.5*7.5 rectangular shape container which can resist hot solution was used). XG (xanthan gum) & LBG (locust bean gum) were weighed out to 0.75 g each and mixed well together before the mixture was dissolved in de-ionized water (DIW) 100 ml. They were then distributed in DIW very well and left to swell for 3-4 hours.

A separate foam solution was prepared:

Warmed 25 ml of de-ionized water (about 26 ml to compensate for evaporation) and dissolved 0.125 g of SLS (sodium laurel sulfate), and then suspended 0.075 g of Carbopol while stirring with a magnetic stirrer. Stirring was continued for about 3 hours.

After 3 hours, adjusted pH with Neutral (very tiny amount) to 7 to 7.5 (Change of pH paper: khaki to dark green color), and then put a beaker of the foam solution into an ice-water bath to set the foam. (Neutral is the excipient or ingredient that is used to adjust pH of Carbopol solution and make the solution become very thick. Other alkaline neutralizers can be used.)

Heated the gum solution from step 1 above and stirred on and off, and meanwhile stirred the foam solution from step 3 above with a magnetic stirrer at the highest speed.

Heated the gum solution until it reached 80° C. and then added 5.5 ml PEG400 and stirred for 10 sec.

Removed the magnetic stirrer from the gum solution and poured the foam into the gum solution with a spatula and mixed them together with the spatula.

Poured the gum/foam mixture into each mold and filled it about halfway and then added drug beads and filled up the rest of the mold with the gum/foam mixture and then mixed them quickly before cooling and gelling occured so that the drug beads were homogeneously distributed.

Let it set at room temperature for about 2 to 4 hours.

Put the cooled gel into the refrigerator and left it [usually more than 10 hours (overnight), but variable times for convenience are acceptable].

Took each gel out of the container and placed on waxed or plastic sheeting.

Dried the gels in a laboratory vacuum oven at 53° C. for 4.5 to 5 hours. The exact vacuum, temperature, and drying times are all variable depending on the equipment available. These conditions gave good results using a water vacuum.

### Example 20

This section concerns the production of a sustained release formulation of hydrochlorothiazide.

1. Sugar spheres of size 18-20 mesh were layered with hydrochlorothiazide suspension. The suspension was prepared by suspending 9 grams of PVP (Povidone K-30), 3 grams of Klucelg (HPC) 3 g (both are used as binders) and 40 grams of HCTZ in 100 ml of de-ionized water at room temperature overnight.

2. Layering was performed in a bottom spraying, Wurster column, spray-coating chamber.

TABLE 13
Conditions for the spray coating:

| | Unit |
|---|---|
| Inlet temperature (° C.) | 55-60 |
| Air pressure (psi) | 18 |
| Nozzle for drug layering (mm) | 1.0 |
| Nozzle for sustained release coating (mm) | 1.0 |

3. HCTZ layered spheres were coated with suspension of Surelease and Opadry mixture. Drug layered spheres 100 g were coated with the suspension of 1 g Opadry and 8.06 g Surelease in 10 ml de-ionized water. Total percent of coating applied on HCTZ layered spheres was 3% which consisted of 66.6% Surelease and 33.3% Opadry.

4. After layering was complete, spheres were dried in the chamber for approximately 30 minutes.

### Example 21

This section concerns the administration of GRDs containing hydrochlorothiazide to human subjects.

Two formulations for hydrochlorothiazide (an immediate release formulation (IR) and a gastric retention device (GRD)) containing sustained release formulations (SR) were administered in the bio-study (bioavailability study). A commercial tablet containing 50 mg of HCTZ was used as an IR control, and spray-coated beads equivalent to 50 mg of HCTZ were formulated for SR in the lab. The process of SR formulation is described above. Bio-study was performed to evaluate the bioavailability as well as pharmacodynamics of HCTZ from a GRD compared to those from an IR.

Monitoring concentrations of hydrochlorothiazide in the urine of healthy adult volunteers allowed comparison of the relative bioavailability of hydrochlorothiazide from the GRD formulation and from a conventional tablet. Participation involved at least two days for each treatment with at least 72-hours washout period between doses. An IR was given once and the GRD was repeated twice to test the reproducibility of the new dosage form, GRD. A 50 mg dose of was chosen for the study because it was in the range of the recommended dose from the PDR (Physician's Desk References) and it produced concentrations high enough to make HPLC analysis efficient. Six subjects participated in the study, 4 healthy males and 2 healthy females. They were not allowed any food or drink containing caffeine, nor alcohol or other medications. Smokers and vegetarians were not included. Subjects fasted overnight and at least 2 hours following dosing. They voided their bladder before receiving a single dose of hydrochlorothiazide in each study and took the dose with 12 ounces of water. After dosing, subjects received a set of containers in which to collect their urine and a time sheet on which to record the time of urination. Subjects collected all urine within a 24-hour period after oral administration of the formulations. Urine samples were collected during the period 0-1,1-2, 2-3, 3-4, 4-6, 6-8, 8-10, 10-12, 12-24, 24-36, and 36-48 hours. Urine samples were refrigerated until delivered to the researcher. The volume of urine collected was measured in order to calculate total amount of drug recovered. A modified method for HPLC (High performance Liquid Chromatography) assay of Papadoyannis et al. (1998) was used to analyze small portions of urine samples for the drug content.

### Example 22

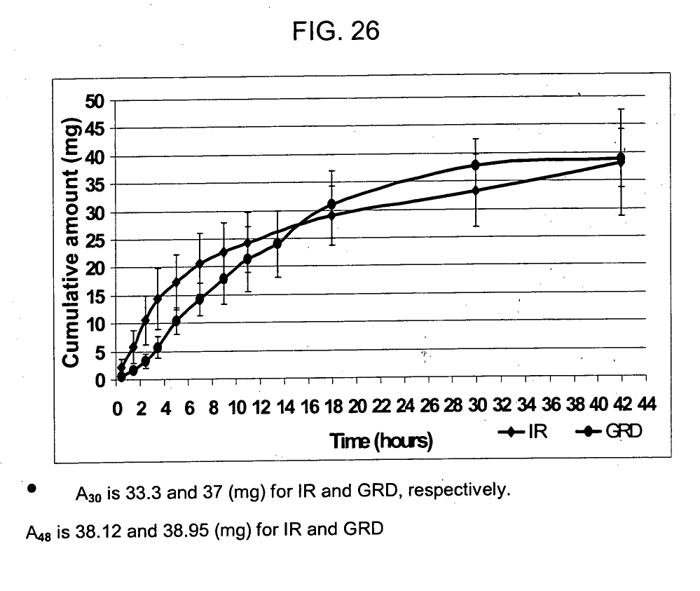

This section concerns the analysis of pharmacokinetic parameters and urine output data following administration of GRDs containing hydrochlorothiazide. GRDs containing the drug, hydrochlorothiazide, were administered to human subjects as outlined in Example 21. Average pharmacokinetic parameters for each treatment under fasting conditions are provided in the following Table 14, and FIG. 26 shows cumulative amount of drug excreted vs. time. Elimination half-life (t_{1/2}) was approximately 7 hours. The values of A₀₋₃₆ were compared for statistical analysis because it was not possible to obtain the value at 48 hours for an IR from one subject due to the short half-life.

### Example 23

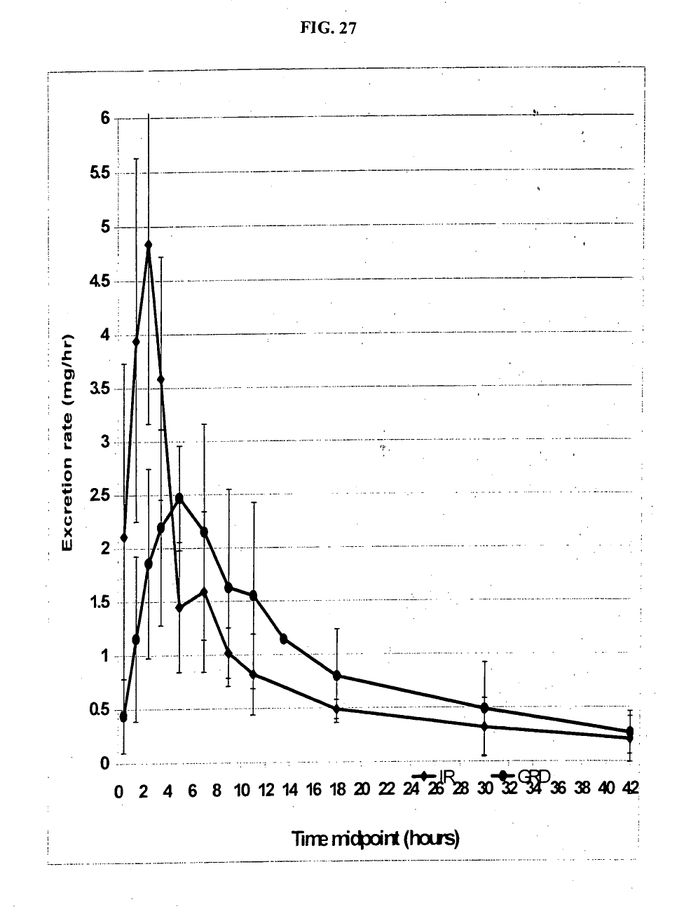

This example concerns the effects of GRD administration of hydrochlorothiazide to fasting subjects. GRDs containing the drug, hydrochlorothiazide, were administered to human subjects as outlined in Example 21 and average pharmacokinetic parameters for each treatment were analyzed as outlined in Example 23. Mean A₀₋₃₆ₕ from IR (33.3 mg, 66.6%) was found to be significantly different (P<0.05) relative to that from GRD (37 mg, 75.4%) in fasting conditions, although the difference is less than 10%. A difference less than 20% is generally considered to be insignificant from FDA BA/BE guidance. From FIG. 26 and Table 14, mean values for total drug absorbed and collected in the urine were equivalent, (A_{0-48\}) were 38.12 mg (76.2%) and 38.95 mg (77.9%) for IR and GRD in fasting conditions, respectively. A₀₋₄₈ was based on assuming 50% of absorbed dose appears intact in the urine. Thus, the GRD resulted in essentially the same amount of drug being absorbed as from an IR up to 48 hours in fasting subjects. However, as illustrated in Tables 14 and 15, the effects on urinary excretion were surprisingly quite different. Specifically, Tables 14, 15 and FIG. 27 demonstrate that a higher maximum excretion rate of drug (Rmax) occurred at an earlier time (tₘₐₓ) from the immediate release (IR) capsule than that from the new formulation (GRD) (4.84 mg/hr at 2.5 hr vs 2.5 mg/hr at 5 hr).

TABLE 14
Pharmacokinetic parameters and Urinary output data for IR:

| AVG (IR) | | | | | | | |
|---|---|---|---|---|---|---|---|
| Mid | Excretion | | | | | | |
| time | Rate | | | Cum. Vol | Water | Water | Ratio of |
| point | (mg/hr) | Vol/Time | Cum.Amt | (ml) | intake (ml) | intake/hr | output/input |
| 0.5 | 2.109834 | 270.3333 | 2.109834 | 257.5 | 355 | 355 | 0.725352113 |
| 1.5 | 3.94137 | 363.4618 | 5.860838 | 628.333 | 710 | 355 | 0.884976526 |
| 2.5 | 4.838802 | 311.3587 | 10.61732 | 940 | 1098.33333 | 388.333333 | 0.855842185 |
| 3.5 | 3.587672 | 310.75 | 14.43554 | 1204.67 | 1486.66667 | 388.333333 | 0.810313901 |
| 5 | 1.44891 | 323.8361 | 17.30009 | 1856.6 | 1866 | 189.666667 | 0.994962487 |
| 7 | 1.59156 | 284.3054 | 20.60636 | 2281.33 | 2398.33333 | 266.166667 | 0.951216122 |
| 9 | 1.017416 | 206.9508 | 22.53981 | 2628 | 3023.33333 | 312.5 | 0.86923925 |
| 11 | 0.816937 | 145.1 | 24.34742 | 3006.33 | 3496.66667 | 236.666667 | 0.859771211 |
| 18 | 0.489007 | 108.4572 | 29.01896 | 4535 | 4315.83333 | 68.2638889 | 1.050782004 |
| 30 | 0.316279 | 79.62282 | 33.26785 | 5560.83 | 5617.5 | 108.472222 | 0.989912476 |
| 42 | 0.204944 | 81.23696 | 38.12199 | 6068.67 | 6013.33333 | 32.9861111 | 1.009201774 |

TABLE 15
Pharmacokinetic parameters and Urinary output data for GRD:

| AVG (GRD) | | | | | | | |
|---|---|---|---|---|---|---|---|
| Mid | Excretion | | | | | | |
| time | Rate | | | Cum. Vol | Water | Water | Ratio of |
| point | (mg/hr) | Vol/Time | Cum.Amt | (ml) | intake (ml) | intake/hr | output/input |
| 0.5 | 0.438872 | 214.8434 | 0.483118 | 200.556 | 355 | 355 | 0.564945227 |
| 1.5 | 1.155346 | 379.355 | 1.708467 | 529.273 | 603.75 | 248.75 | 0.876642198 |
| 2.5 | 1.86002 | 367.5 | 3.304097 | 942.909 | 916.818182 | 313.068182 | 1.028458106 |
| 3.5 | 2.195914 | 390.6247 | 5.698541 | 1311.55 | 1254.58333 | 337.765152 | 1.045403219 |
| 5 | 2.46914 | 356.0098 | 10.43505 | 1927.67 | 1820.45455 | 282.935606 | 1.05889305 |
| 7 | 2.151739 | 317.013 | 14.18478 | 2592.25 | 2484.16667 | 331.856061 | 1.04350889 |
| 9 | 1.627401 | 264.23 | 17.92668 | 2975.18 | 3062.91667 | 289.375 | 0.971355783 |
| 11 | 1.552815 | 276.3023 | 21.32656 | 3598.92 | 3612.08333 | 274.583333 | 0.996354828 |
| 13.5 | 1.144381 | 214.9586 | 24.04283 | 4217.29 | 4307.29167 | 231.736111 | 0.9791052 |
| 18 | 0.79889 | 114.8045 | 31.00009 | 5003.18 | 4972.91667 | 73.9583333 | 1.006085996 |
| 30 | 0.487425 | 100.6936 | 37.71256 | 6378.5 | 6481.66667 | 125.729167 | 0.984083312 |
| 42 | 0.265091 | 93.9158 | 38.95911 | 7466.78 | 7380 | 74.8611111 | 1.011758506 |

### Example 24

This section concerns the profile for HCTZ-50 mg over 48-hours in fasting subjects. GRDs containing the drug, hydrochlorothiazide, were administered to human subjects as outlined in Example 21 and average pharmacokinetic parameters for each treatment were analyzed as outlined in Example 23. The cumulative amount of HCTZ-50 mg vs. time was analyzed as outlined in Example 23.

Cmax and Tmax is 4.84 and 2.46 (mg/ml), and 2.5 and 5 (hr) for IR and GRD, respectively.

T_{1/2} is 7 hours.

The rate of urine production was similar in both IR and GRD up to 10 hours post-dosing. This is quite unexpected since the amount of drug absorbed and drug concentrations in the body are less from the GRD revealed herein compared to the commercial IR capsule. And, diuresis started decreasing for the IR capsule after 10 hours, whereas a high amount of diuresis was maintained for GRD for a longer time period.

The initial equal amount of diuresis is surprising since less drug is absorbed initially from the GRD (Rmax 4.8 (μg/ml) at tₘₐₓ, 2.5 hours and 2.5 (μg/ml) at tₘₐₓ, 5 hours in fasting condition for IR and GRD, respectively) which now teaches that less drug can be more effective which is not common for drugs. In fact, if less amount of drug is input, less effect is expected but the opposite effect occurred with this new GRD and the diuretic.

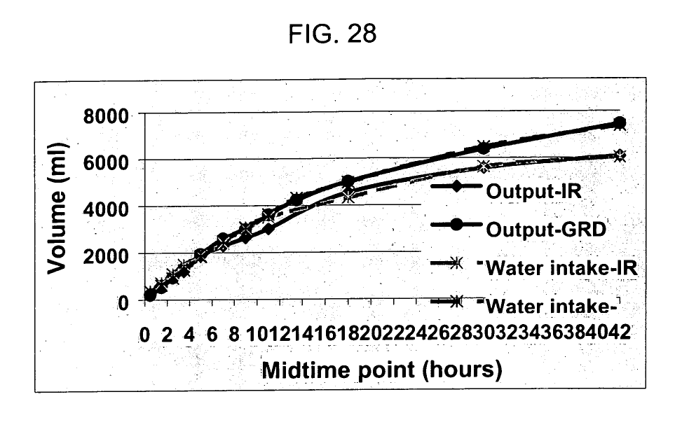

It also was clearly observed that drug effect on urine production from GRD was continuous until approximately 15 hours (see data provided in the table above). From FIG. 28, a comparison between urine production and water-intake, and between the ratio of urine production and water-intake were studied and the cumulative amount of urine output from hydrochlorothiazide in both IR and GRD is consistent with water-intake.

Increasing body fluid excretion in healthy, normal subjects stimulated water-intake. Total amount of urine production was higher from the same dose in a GRD compared to IR, which can be attributed to prolonged drug input from GRD followed by a feedback increased amount of water-intake to compensate for the unexpected increased drug effect.

This overall increased effect is also surprising (in addition to the initial greater effect with a smaller drug input discussed above) since it is well known that in order to increase diuretic effect it is necessary to increase the drug dose. In fact, most drug response curves are log-linear which means that usually an increase in effect is less (smaller percentage) than the increase in dose after an initial response threshold is crossed. But, in this case, the bioavailability of drug under fasting conditions was essentially equal, but the diuretic effect was increased 27% as shown by FIG. 38 and the table provided above.

Results from this bioavailability study of hydrochlorothiazide establishes that the device was retained long enough to release all or most drug in the stomach, but also that the dosage form controlled drug release to prolong drug effect. Thus, the GRD is an excellent device for administering hydrochlorothiazide as well as other diuretics that exhibit limited absorption sites in the upper part of the intestine. This dosage form can improve patient care by avoiding high drug peak concentrations that may induce undesirable side effects (see side effects information below), increase drug effect per dose administered, and achieving prolonged drug effect.

### Example 25

This section concerns the side effects in human subjects following administration of hydrochlorothiazide in a GRD. GRDs containing the drug, hydrochlorothiazide, were administered to human subjects as outlined in Example 21, and the following side effects were reported:

Three out of 7 subjects reported side effects from an IR dosage form between 4-6 hours post-dosing.

Adverse reactions reported were severe or moderate headache, dehydration, and fatigue.

One subject did not continue in the study due to severe headache, dehydration, and fatigue.

No adverse reactions were reported from the same dose of hydrochlorothiazide in a GRD.

Subjects were encouraged to drink more water after the 1st study with an IR due to awareness of the consequence of dehydration from HCTZ.

### Example 26

This example concerns methods for varying physical and drug release characteristics of a GRD by using different gel dehydration conditions. Gastric retention devices comprising a gel formed from a polysaccharide were prepared as follows:

### Preparation of Gastric Retention Formulation

1. Before beginning, all ingredients and molds were gathered;

2. 0.75 g locust bean gum was added to 100 ml DIW with continuous mixing followed by 0.75 g xanthan gum (slowly sprinkled a small amount of gum on the surface of water, then mixed well before adding another portion);

3. the gum suspension formed in step 2 was allowed to swell fully for 2 hours;

4. a foam solution was prepared by warming 25 ml DIW to about 50° C. then dissolving 0.125 g sodium lauryl sulfate. Suspended 0.075 g Carbopol 934 and stirred rapidly with a magnetic stirrer for 2 hours;

5. pH of the foam solution was adjusted from 4 with 1 N NaOH to 7-7.5; the pH 7-7.5 foam solution was placed into an ice bath to set the foam, with continued rapid stirring;

6. the gum mixture from step 3 above was heated to 80-85° C. followed by adding 5 ml PEG 400;

7. the foam solution was poured into the gum mixture and mixed well;

8. accurately weighed HCTZ powder was added to the mixture from 7.

9. the mixture was heated, if necessary, until pourable and then poured into suitable molds;

10. the molds were allowed to stand at room temperature for 2 hours;

11. the molds were refrigerated 2-18 hours;

12. each film was then removed from the mold and held at −80° C. for 2 hours;

13. each film was then freeze dried for 16-18 hours.

Ingredients list

| | | |
|---|---|---|
| Xanthan gum | 0.75 | g |
| LBG | 0.75 | g |
| SLS | 0.125 | g |
| Carbopol 934 | 0.075 | |
| PEG 400 | 5 | ml |

Gels from step 11 above also were vacuum oven dried at 50-55 ° C. as described in earlier examples. Drying produced flexible, soft films, which were easy to roll and insert into capsules. The gels typically were placed on the drying tray such that the height of the wet gel was about 1 cm before drying. After drying, the texture of the resultant films, as well as the shape and size, were dependent upon the vacuum and temperature. With freeze drying, for example, there is little or no change in either the shape or size of the starting material, but the surface texture and internal structure of the material may be different from the starting material. Thus, with freeze drying, the film produced following dehydration was typically of the same size and shape as the starting material. That is, if the initial gel was sized to be 7.5×1.5×1.0 cm, then the freeze dried product was also about 7.5×1.5×1.0 cm.

The method of dehydration affects not only the size and shape of the resultant film but was also shown to affect the release pattern of drug incorporated into the gastric retention device. Hydrochlorothiazide powder was incorporated into gels of the formulation described above such that dimensions of 7.5×1.5×1.0 cm contained 50 milligrams of drug, and then these compositions were either vacuum oven dried or freeze dried. The resultant films were compressed by rolling and twisting and inserted into gelatin capsules.

When dissolution studies were conducted in simulated gastric fluid using GRDs containing hydrochlorothiazide, the drug was released relatively quickly from the vacuum oven dried GRD. However, release of the drug from the freeze dried GRD was relatively slow, with approximately only 50 percent of the drug released in four hours and about only 80 percent of the drug released in eight hours and complete release of drug in approximately 20-24 hours.

After the gelatin capsules dissolves and the GRD has been exposed to gastric fluid for variable time periods such as from about 2 to about 4 hours that the freeze dried product retains a relatively more rigid and stronger texture than the vacuum dried product at comparable time periods as determined by tactile measurements. It was also observed that the freeze dried product expands somewhat more rapidly after exposure to gastric fluid than the vacuum oven dried product. In one case, for example, after immersion in simulated gastric fluid the freeze dried product hydrated and expanded in 25 to 35 minutes, but the vacuum dried product took 45 to 50 minutes to rehydrate to the same extent.

These results show that methods of dehydration can be utilized to affect physical and drug release characteristics for gastric retention devices. In some experiments release rate was studied when drug-containing gel was either molded or cut to a specific size prior to freeze drying as individual units, and compared to drug release when drug containing gel was freeze dried as sheets and then cut into the proper size after drying. In these embodiments, the release rate of drug after hydration of the GRD in simulated gastric fluid was essentially the same for all three methods of preparation.

### Example 27

This section concerns the ability of relatively small gastric retention devices to be retained in the stomach for prolonged periods. Hydrochlorothiazide powder was incorporated into gels of the formulation described in Example 26, such that dimensions of 3.5×1.5×1.0 cm or 5.5×1.5×1.0 cm contained 50 milligrams of drug, the gels were freeze dried, and resultant films were compressed by rolling and twisting and inserted into gelatin capsules. ‘0’ size capsules were used for the smaller GRD (SGRD) and ‘00’ capsules were used for the other GRD (termed “intermediate gastric retention device” IGRD in this study).

The SGRD and IGRD and an immediate release tablet (IR) containing 50 mg. each of hydrochlorothiazide were tested in 12 healthy volunteers (five females and seven males). Subjects ranging in age between 26 and 43 years and weighing between 45 and 117 kg were treated. In each phase of the study each of the subjects received 50 milligrams hydrochlorothiazide in the form of either conventional immediate release tablets, IGRD, or SGRD in a randomized crossover fashion with a washout period of at least 4 days. There were two phases: fed subjects and fasted subjects. All subjects fasted overnight (10 hours or longer). They were then given a standard breakfast immediately before the treatment dose with 200 ml of water and no more food allowed for the next two hours (fed subjects), or were given the drug on an empty stomach, and then fed a standard breakfast two hours later. The standard breakfast was a sausage, biscuit, egg, and 240 ml orange juice from Burger King.

All urine was collected from all subjects for 72 hours and evaluated pharmacokinetically for hydrochlorothiazide absorption and excretion. The drug was absorbed more slowly and for a prolonged time period from both the SGRD and the IGRD compared to the IR dosage form. Some average information from the drug excretion in urine versus time curves and deconvolution of the data are recorded in Table 16.

TABLE 16
Averaged hydrochlorothiazide excretion absorption data

| FASTED | | | | FED | | |
|---|---|---|---|---|---|---|
| | IR | IGRD | SGRD | IR | IGRD | SGRD |
| Max ER | 6.44 | 2.1 | 2.1 | 6.7 | 2.2 | 1.8 |
| Tp | 2.3 | 4.1 | 3.2 | 2.8 | 6.6 | 6.7 |
| Drug recovered | 25.4 | 23.4 | 17.9 | 31.5 | 27.4 | 21.7 |
| Input time (hr) | 3 | 26 | 12 | 3 | 27 | 14 |

Max ER = maximum average drug excretion rate (mg/hr);
Tp = time to Max ER,
Drug recovered in urine is in mg;
Input time = length of time drug continued to be absorbed as determined by deconvolution

These data demonstrate prolonged absorption of hydrochlorothiazide from both the IGRD and SGRD. Because hydrochlorothiazide is a drug with a window of absorption in the upper portion of the small intestine it can be concluded that the IGRD and SGRD both were retained in the stomach for surprisingly long time periods on both the fasted and fed stomach.

### Example 28

Hydrochlorothiazide (HCTZ) is a thiazide diuretic that is recommended as a first line agent in hypertension. HCTZ is only absorbed from the upper part of the duodenum and once it passes this absorption site, little or no absorption takes place. This example demonstrates that formulation of HCTZ as a swellable gastric retention device (GRD) as described above results in gastric retention for extended time periods. One formulation stayed in the stomach for 10-18 hours, providing continuous drug input for that length of time. Such a drug release profile reduces blood pressure in hypertensive patients and reduces fluctuations in blood pressure during the day.

Methods:

6 adult mildly hypertensive subjects currently receiving medication for their hypertension participated in this study. Each subject was given an automatic blood pressure monitor (Omron HEM-637, Omron Healthcare, Inc., Ill.) and asked to measure blood pressure in the sitting position four times a day: when he or she wakes up, two hours after they take the dose, before dinner and at bedtime. The subjects were asked to continue taking their current medications while monitoring their blood pressure for 3 days without study treatment interventions. The subjects were then randomly assigned to receive either 25 mg HCTZ once a day after breakfast using either either commercially available immediate release tablets or the new gastric retention formulation for 7 days. The subjects were then crossed over to receive the other treatment for seven more days. If the subjects were already on any dose of HCTZ they were asked to stop taking it after baseline monitoring and continue after the study ended. The patients were instructed to continue any other medication they were taking other than the diuretic throughout the study period.

At the end of each treatment phase, the subjects were given a short questionnaire asking them to rate any side effects they may have experienced and to report any change in nocturnal habits and interference with their social and academic lives while in the study.

Results and Discussion:

In three of the subjects who have completed the study thus far, there was a distinct decrease in fluctuation of blood pressure readings during the day when the subjects were using the GRD formulation. The GRD achieved blood pressure decreases comparable to or more than those produced by the IR tablets in all subjects.

While on the IR tablets, three subjects reported headaches, two reported urinary frequency and four reported urgency of urination, and two reported unusual thirst and dry mouth. After taking the GRD, one subject reported a headache, one reported urinary frequency and three reported urgency of urination, one subject reported muscle aches and 2 reported dry mouth. On a scale of 0-10, side effects while taking GRD were rated milder than IR tablets. No subjects reported changes in nocturnal habits or interference with social and/or academic life while taking either formulation.

It is known that the full effect blood pressure modulation by HCTZ cannot be evaluated before 4-6 weeks of treatment. This study was designed to compare the initial blood pressure lowering effects and side effects of HCTZ when given as IR tablets or GRD early in treatment and not to evaluate the full efficacy of HCTZ as a hypertensive agent.

It is concluded that GRD embodiments successfully provide continuous input of HCTZ over several hours and longer than the immediate release tablets, which was reflected as decreased blood pressure fluctuations over the day in three subjects. HCTZ in GRD embodiments also is successful in controlling blood pressure in Stage 1 hypertension as well as or better than HCTZ IR tablets in the early days of treatment. The GRD has been given to humans in a multiple dosing regimen and no GI side effects were reported by any of the test subjects.

### Example 30

This example describes the incorporation of lipid material into embodiments of the disclosed GRD. GRDs containing lipid material are useful for further influencing gastric emptying and appetite. The upper small intestine contains receptors known to close the pyloric sphincter and decrease rate of gastric emptying when stimulated by lipids. Long chain fatty acids and other fats have been shown to slow gastric emptying through stimulation of the fat receptors in the duodenum.

This example demonstrates the surprising result that fatty/oily materials can be incorporated into hydrophilic gels, such as are used in the disclosed GRDs. In this example, olive oil or sodium myristate was added to the gelling ingredients before gelation occurred. These lipophilic materials did not substantially interfere with gelation.

Methods:

25 ml DIW was warmed to about 50° C. and then 0.125 g SLS was dissolved followed by 0.075 g Carbopol 934, and the mixture was stirred rapidly for 2 hours to form a dense foam. The foam was then neutralized using 1 N NaOH and cooled in an ice bath.

0.75 g of LBG was dispersed in 100 ml DIW followed by 0.75 g XG. The gums were allowed to fully swell for 2 hours before heating to 85° C. 5 ml PEG 400 was then added to the gum mixture with stirring.

The foam solution was added to the gum mixture and the resulting product was again warmed until it became pourable. The mixture was then poured into suitable molds or trays and left to cool at room temperature for 2 hours.

Prepared formulations were refrigerated overnight, then cut into suitable sizes, individually frozen at −80° C. for 1-2 hours, and finally freeze dried for 16-20 hours.

In the formulation using sodium myristate, 4 grams of sodium myristate was added to the foam solution before neutralization and mixed well to produce a final product containing 0.25 grams Na myristate/GRF.

In the formulation including olive oil, 16 ml of olive oil was mixed with the gums after addition of PEG 400 and before the foam solution was folded in. Concentration of olive oil in each formulation was about 1 ml.

Results:

Addition of sodium myristate yielded a very rigid product that was difficult to flatten and fold into a capsule. When rehydration of this product was attempted, the GRD exhibited hydrophobic characteristics and would not unfold.

Olive oil, on the other hand, produced a very flexible product. During flattening and folding some of the oil was squeezed out of the GRF. Of course, less oil or sodium myristate can be used in the formulation. Rehydration of the GRF containing olive oil occurred at approximately the same rate as controls without olive oil. Cottonseed and other oils also can be incorporated in the GRF. These formulations are useful for producing a sensation of being full in a subject trying to lose weight or any condition where it is desirable to delay stomach emptying, such as with a hyperactive stomach or for delivery of agents in the stomach for local action in the stomach or for slow delivery to the upper small intestine. This is an example of a gastric retention device that expands sufficiently in the stomach of a subject to at least partially suppress appetite in the subject, said suppression being due to the device or the oil released from the device, or a combination of both. And, they are useful for formulation of lipophilic drugs with limited solubility in water.

It will be apparent to those of ordinary skill in the art that the precise details of the methods described may be varied or modified without departing from the spirit of the described invention. We claim all such modifications and variations that fall within the scope and spirit of the claims below.

I claim:

1. A gastric retention device comprising a gel formed from a polysaccharide, the device being formed to a size suitable for administration to a subject.

2. The gastric retention device of claim 1 having a coating applied to an outer surface thereof or housed within an ingestible capsule.

3. The gastric retention device of claim 2 where the coating or capsule is erodible by gastric fluid.

4. The gastric retention device of claim 2 where the coating or capsule is an enteric coating.

5. The gastric retention device of claim 1 where the polysaccharide comprises xanthan gum.

6. The gastric retention device of claim 1 where the polysaccharide comprises locust bean gum.

7. The gastric retention device of claim 1 where the polysaccharide comprises a mixture of xanthan gum and locust bean gum.

8. The gastric retention device of claim 1 further comprising a material selected from the group consisting of a plasticizer, a pH adjuster, a GI motility adjuster, a viscosity adjuster, a therapeutic agent, a diagnostic agent, an imaging agent, an expansion agent, a surfactant, and mixtures thereof.

9. The gastric retention device of claim 1 compressed to a size suitable for oral administration.

10. The gastric retention device of claim 1 where administration comprises oral administration, rectal administration, vaginal administration, nasal administration, or administration in the oral cavity.

11. The gastric retention device of claim 1 which expands following administration and where, following expansion, the device is a cube, a cone, a cylinder, a pyramid, a sphere, a column, or a parallelepiped.

12. The gastric retention device of claim 8 where the diagnostic or therapeutic agent is selected from the group consisting of nucleic acids, proteins, and combinations thereof.

13. The gastric retention device of claim 1 further comprising a material selected from the group consisting of AIDS adjunct agents, alcohol abuse preparations, Alzheimer's disease management agents, amyotrophic lateral sclerosis therapeutic agents, analgesics, anesthetics, antacids, antiarythmics, antibiotics, anticonvulsants, antidepressants, antidiabetic agents, antiemetics, antidotes, antifibrosis therapeutic agents, antiftngals, antihistamines, antihypertensives, anti-infective agents, antimicrobials, antineoplastics, antipsychotics, antiparkinsonian agents, antirheumatic agents, appetite stimulants, appetite suppressants, biological response modifiers, biologicals, blood modifiers, bone metabolism regulators, cardioprotective agents, cardiovascular agents, central nervous system stimulants, cholinesterase inhibitors, contraceptives, cystic fibrosis management agents, deodorants, diagnostics, dietary supplements, diuretics, dopamine receptor agonists, endometriosis management agents, enzymes, erectile dysfumction therapeutics, fatty acids, gastrointestinal agents, Gaucher's disease management agents, gout preparations, homeopathic remedys, hormones, hypercalcemia management agents, hypnotics, hypocalcemia management agents, immunomodulators, immunosuppressives, ion exchange resins, levocarnitine deficiency management agents, mast cell stabilizers, migraine preparations, motion sickness products, multiple sclerosis management agents, muscle relaxants, narcotic detoxification agents, narcotics, nucleoside analogs, non-steroidal anti-inflammatory drugs, obesity management agents, osteoporosis preparations, oxytocics, parasympatholytics, parasympathomimetics, phosphate binders, porphyria agents, psychotherapeutic agents, radio-opaque agents, psychotropics, sclerosing agents, sedatives, sickle cell anemia management agents, smoking cessation aids, steroids, stimulants, sympatholytics, sympathomimetics, Tourette's syndrome agents, tremor preparations, urinary tract agents, vaginal preparations, vasodilators, vertigo agents, weight loss agents, Wilson's disease management agents, and mixtures thereof.

14. The gastric retention device of claim 8 where the diagnostic or therapeutic agent is provided by a tablet, capsule, powder, bead, pellet, granules, solid dispersion, or combinations thereof.

15. The gastric retention device of claim 8 where the diagnostic or therapeutic agent is more soluble in gastric fluid than intestinal fluid.

16. The gastric retention device of claim 8 where the diagnostic or therapeutic agent is more soluble in intestinal fluid than gastric fluid.

17. The gastric retention device of claim 8 where the diagnostic or therapeutic agent is absorbed better within small intestine than within large intestine.

18. The gastric retention device of claim 8 where the diagnostic or therapeutic agent is absorbed better within stomach than within intestines.

19. The gastric retention device of claim 8 where the diagnostic or therapeutic agent is absorbed better within intestines than within stomach.

20. The gastric retention device of claim 8 where the diagnostic or therapeutic agent is abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, carvedilol, caspofungin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fluticasone propionate, furosemide, hydrochlorothiazide/triamterene, lamivudine, lamotrigine, lithium carbonate, losartan potassium, melphalan, mercaptopurine, mesalazine, mupirocin calcium cream, nabumetone, naratriptan, omeprazole, ondansetron hydrochloride, ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine or lamivudine, or mixtures thereof.

21. A gastric retention device, comprising a compressed device that, upon ingestion by a subject, expands sufficiently, and is sufficiently robust upon expansion, to preclude passage of the device through the subject's pylorus for a predetermined time up to 24 hours while still allowing food to pass.

22. The gastric retention device according to claim 21, further comprising a therapeutic or diagnostic agent that is absorbed more gastrically than intestinally.

23. The gastric retention device of claim 21 having an expansion coefficient of at least 3.0.

24. The gastric retention device of claim 21 having an expansion coefficient of at least 6.0.

25. The gastric retention device according to claim 21 having an expansion coefficient of at least 8.0.

26. A gastric retention device formed from a mixture comprising a sugar, a polysaccharide, or combinations thereof.

27. The gastric retention device according to claim 1 where the gel is a thermally induced gel.

28. The gastric retention device according to claim 1 where the gel is a chemically induced gel.

29. The gastric retention device according to claim 1 and further comprising hydrochlorothiazide, ranitidine HCI, or amoxicillin.

30. The gastric retention device according to claim 21 and further comprising hydrochlorothiazide, ranitidine HCl, or amoxicillin.

31. The gastric retention device according to claim 21 further comprising enzymes that aid erosion of the coating, capsule or device following ingestion of the device.

32. A gastric retention device, comprising:
a compressed device that, upon ingestion by a subject, expands sufficiently, and is sufficiently robust upon expansion, to preclude passage of the device through the subject's pylorus for a predetermined time up to 24 hours while still allowing food to pass, the compressed device further comprising a material selected from the group consisting of a plasticizer, a pH adjuster, a GI motility adjuster, a viscosity adjuster, a therapeutic agent, a diagnostic agent, an expansion agent, a surfactant, and mixtures thereof; and
a coating erodible by gastric fluid applied to an outer surface of the compressed device or a capsule erodible by gastric fluid housing the compressed gel.

33. An expandable gastric retention device prepared from a mixture comprising xanthan gum and locust bean gum, the device being compressed to form a compressed device, the compressed device having a coating applied to an outer surface thereof or being housed in a capsule erodible by gastric fluid.

34. The gastric retention device of claim 33 wherein the device is substantially dehydrated.

35. The gastric retention device of claim 33 wherein the device is freeze-dried.

36. The gastric retention device of claim 33 having an expansion coefficient of at least 3.0.

37. The gastric retention device of claim 33 having a weight ratio of xanthan gum to locust bean gum of from about 1:4 to about 4:1.

38. The gastric retention device of claim 33 having a weight ratio of xanthan gum to locust bean gum of about 1:1.

39. The gastric retention device of claim 33 further comprising a material selected from the group consisting of a plasticizer, a pH adjuster, a GI motility adjuster, a viscosity adjuster, a therapeutic agent, a diagnostic agent, an expansion agent, a surfactant, and mixtures thereof.

40. The gastric retention device of claim 39 where the plasticizer is polyethylene glycol.

41. The gastric retention device of claim 39 where the pH adjuster is sodium phosphate or disodium phosphate.

42. The gastric retention device of claim 39 where the expansion agent is sodium lauryl sulfate.

43. The gastric retention device of claim 39 where the viscosity adjuster is Carbopol.

44. The gastric retention device of claim 39 where the viscosity adjuster is polyvinyl pyrrolidone.

45. The gastric retention device of claim 33 where, following expansion, the device is a cube, a cone, a cylinder, a pyramid, a sphere, a column, or a parallelepiped.

46. The gastric retention device of claim 33 having a weight ratio of xanthan gum to locust bean gum of from about 1:4 to about 4:1, and further comprising a material selected from the group consisting of Carbopol, sodium lauryl sulfate, PEG400, and mixtures thereof.

47. The gastric retention device of claim 46 having a weight ratio of xanthan gum to locust bean gum of about 1:1.

48. The gastric retention device of claim 33 further comprising a material selected from the group consisting of a diagnostic agent, a therapeutic agent, and mixtures thereof.

49. The gastric retention device of claim 48 where the agent is selected from the group consisting of nucleic acids, proteins, AIDS adjunct agents, alcohol abuse preparations, Alzheimer's disease management agents, amyotrophic lateral sclerosis therapeutic agents, analgesics, anesthetics, antacids, antiarythmics, antibiotics, anticonvulsants, antidepressants, antidiabetic agents, antiemetics, antidotes, antifibrosis therapeutic agents, antifungals, antihistamines, antihypertensives, anti-infective agents, antimicrobials, antineoplastics, antipsychotics, antiparkinsonian agents, antirheumatic agents, appetite stimulants, appetite suppressants, biological response modifiers, biologicals, blood modifiers, bone metabolism regulators, cardioprotective agents, cardiovascular agents, central nervous system stimulants, cholinesterase inhibitors, contraceptives, cystic fibrosis management agents, deodorants, diagnostics, dietary supplements, diuretics, dopamine receptor agonists, endometriosis management agents, enzymes, erectile dysfimction therapeutics, fatty acids, gastrointestinal agents, Gaucher's disease management agents, gout preparations, homeopathic remedies, hormones, hypercalcemia management agents, hypnotics, hypocalcemia management agents, immunomodulators, immunosuppressives, ion exchange resins, levocarnitine deficiency management agents, mast cell stabilizers, migraine preparations, motion sickness products, multiple sclerosis management agents, muscle relaxants, narcotic detoxification agents, narcotics, nucleoside analogs, non-steroidal anti-inflammatory drugs, obesity management agents, osteoporosis preparations, oxytocics, parasympatholytics, parasympathomimetics, phosphate binders, porphyria agents, psychotherapeutic agents, radio-opaque agents, psychotropics, sclerosing agents, sedatives, sickle cell anemia management agents, smoking cessation aids, steroids, stimulants, sympatholytics, sympathomimetics, Tourette's syndrome agents, tremor preparations, urinary tract agents, vaginal preparations, vasodilators, vertigo agents, weight loss agents, Wilson's disease management agents, and mixtures thereof.

50. The gastric retention device of claim 48 where the agent is provided by a tablet, capsule, powder, bead, pellet, granules, solid dispersion, or combinations thereof.

51. The gastric retention device of claim 48 where the agent is more soluble in gastric fluid than intestinal fluid.

52. The gastric retention device of claim 48 where the agent is absorbed better by small intestine than by large intestine.

53. The gastric retention device of claim 48 where the agent is hydrochlorothiazide, amoxicillin, or ranitidine HCl.

54. The gastric retention device of claim 33 where the device expands substantially to its final size within 2 hours in an aqueous environment.

55. The gastric retention device of claim 33 where the device expands to 60% of its final size within 2 hours in an aqueous environment.

56. The gastric retention device of claim 33 where the device expands to 80% of its final size within 2 hours in an aqueous environment.

57. The gastric retention device of claim 33 where the device expands substantially to its final size to form an expanded device within 2 hours following ingestion by a subject.

58. The gastric retention device of claim 57 where the size of the expanded device prevents passage of the gastric retention device through a pylorus for a predetermined time.

59. The gastric retention device of claim 57 where the expanded device has at least one dimension greater than a diameter of the pylorus.

60. The gastric retention device of claim 58 where the device allows food passage through the pylorus.

61. The gastric retention device of claim 58 where the device erodes in the presence of gastric fluids and passes through the pylorus after a predetermined time.

62. The gastric retention device of claim 33 where the device substantially remains in the stomach of a subject for at least 2 hours.

63. The gastric retention device of claim 33 where the device substantially remains in the stomach of a subject for at least 9 hours.

64. The gastric retention device of claim 33 where the device substantially remains in the stomach of a subject for at least 24 hours.

65. The gastric retention device according to claim 33 and further comprising enzymes to facilitate gastric erosion of the gel.

66. A gastric retention device capable of remaining in the stomach for at least 24 hours, comprising an expandable device prepared from a mixture comprising (a) carbohydrate gums, and (b) a material selected from the group consisting of a therapeutic, a diagnostic, a plasticizer, a pH adjuster, a GI motility adjuster, a viscosity adjuster, an expansion agent, a surfactant, and mixtures thereof, the device being compressed sufficiently and into a shape suitable for insertion into a gastrically erodible capsule.

67. A gastric retention device capable of remaining in the stomach for at least 9 hours, comprising an expandable device prepared from a mixture comprising (a) xanthan gum and locust bean gum, and (b) a material selected from the group consisting of a therapeutic, a diagnostic, a plasticizer, a pH adjuster, a GI motility adjuster, a viscosity adjuster, an expansion agent, a surfactant, and mixtures thereof, the device being compressed sufficiently and into a shape suitable for insertion into a gastrically erodible capsule.

68. A gastric retention device capable of remaining in the stomach for at least 9 hours, comprising an expandable device prepared from a mixture comprising, by weight, from about 0.1% to about 2.0% xanthan gum, from about 0.1% to about 2.0% locust bean gum, less than 65% polyethylene glycol, less than 1% sodium lauryl sulfate, less than 1% Carbopol by weight, and a biologically effective amount of a therapeutic, a diagnostic, or combinations thereof, the device being compressed sufficiently and into a shape suitable for insertion into a gastrically erodible capsule.

69. A method for making a gastric retention device, comprising:
forming a mixture comprising a polysaccharide;
processing the mixture to form a dried gel in a form suitable for administration to a subject; and
coating the dried gel with a material erodible by gastric fluid or placing the gel into a capsule erodible by aqueous fluid.

70. The method of claim 69 where the mixture comprises locust bean gum.

71. The method according to claim 69 where the mixture comprises xanthan gum.

72. The method of claim 69 where the mixture comprises a polysaccharide, locust bean gum and water.

73. The method of claim 69 where xanthan gum and locust bean gum comprise from about 0.1% to about 65% of the mixture by weight.

74. The method of claim 72 where the mixture further comprises a material selected from the group consisting of a therapeutic agent, a diagnostic agent, a plasticizer, a pH adjuster, a GI motility adjuster, a viscosity adjuster, an expansion agent, a surfactant, and mixtures thereof.

75. The method according to claim 74 where the agent is selected from the group consisting of nucleic acids, proteins, AIDS adjunct agents, alcohol abuse preparations, Alzheimer's disease management agents, amyotrophic lateral sclerosis therapeutic agents, analgesics, anesthetics, antacids, antiarythmics, antibiotics, anticonvulsants, antidepressants, antidiabetic agents, antiemetics, antidotes, antifibrosis therapeutic agents, antifungals, antihistamines, antihypertensives, anti-infective agents, antimicrobials, antineoplastics, antipsychotics, antiparkinsonian agents, antirheumatic agents, appetite stimulants, appetite suppressants, biological response modifiers, biologicals, blood modifiers, bone metabolism regulators, cardioprotective agents, cardiovascular agents, central nervous system stimulants, cholinesterase inhibitors, contraceptives, cystic fibrosis management agents, deodorants, diagnostics, dietary supplements, diuretics, dopamine receptor agonists, endometriosis management agents, enzymes, erectile dysfimction therapeutics, fatty acids, gastrointestinal agents, Gaucher's disease management agents, gout preparations, homeopathic remedys, hormones, hypercalcemia management agents, hypnotics, hypocalcemia management agents, immunomodulators, immunosuppressives, ion exchange resins, levocarnitine deficiency management agents, mast cell stabilizers, migraine preparations, motion sickness products, multiple sclerosis management agents, muscle relaxants, narcotic detoxification agents, narcotics, nucleoside analogs, non-steroidal anti-inflammatory drugs, obesity management agents, osteoporosis preparations, oxytocics, parasympatholytics, parasympathomimetics, phosphate binders, porphyria agents, psychotherapeutic agents, radio-opaque agents, psychotropics, sclerosing agents, sedatives, sickle cell anemia management agents, smoking cessation aids, steroids, stimulants, sympatholytics, sympathomimetics, Tourette's syndrome agents, tremor preparations, urinary tract agents, vaginal preparations, vasodilators, vertigo agents, weight loss agents, Wilson's disease management agents, and mixtures thereof.

76. The method of claim 74 where the mixture further comprises hydrochlorothiazide.

77. The method according to claim 74 where processing comprises freeze-drying the gel.

78. The method according to claim 69 where processing the mixture comprises heating the mixture effectively to thermally induce gelation of the mixture to form a gel.

79. The method according to claim 69 further comprising compressing the dried gel to a size and shape suitable for administration to a subject prior to coating the gel or placing it in a capsule.

80. The method according to claim 74 where the agent is provided by a tablet, capsule, powder, bead, pellet, granule, solid dispersion, or combinations thereof.

81. A method for making a gastric retention device, comprising:
forming a mixture comprising a polysaccharide and a material selected from the group consisting of a plasticizer, a pH adjuster, a GI motility adjuster, a viscosity adjuster, a therapeutic agent, a diagnostic agent, an expansion agent, a surfactant, and mixtures thereof;
heating the mixture to a temperature sufficient to induce gelation of the mixture to form a gel;
drying the gel to form a dried film;
compressing the dried film to form a compressed film; and
coating the compressed film with a material erodible by gastric fluid or placing the gel into a capsule erodible by gastric fluid.

82. The method of claim 81 further comprising incorporating into the gel abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, carvedilol, caspofungin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fluticasone propionate, furosemide, hydrochlorothiazide/triamterene, lamivudine, lamotrigine, lithium carbonate, losartan potassium, melphalan, mercaptopurine, mesalazine, mupirocin calcium cream, nabumetone, naratriptan, omeprazole, ondansetron hydrochloride, ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine or lamivudine, or mixtures thereof.

83. A method for using a gastric retention device, comprising:
providing a gastric retention device; and
administering the gastric retention device to a subject.

84. The method of claim 83 where the gastric retention device further comprises a therapeutic, a diagnostic, or mixtures thereof.

85. The method of claim 83 where the therapeutic or diagnostic is abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, carvedilol, caspofingin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fluticasone propionate, furosemide, hydrochlorothiazide/triamterene, lamivudine, lamotrigine, lithium carbonate, losartan potassium, melphalan, mercaptopurine, mesalazine, mupirocin calcium cream, nabumetone, naratriptan, omeprazole, ondansetron hydrochloride, ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine or lamivudine, or mixtures thereof.

86. The method according to claim 83 where the gastric retention device comprises an expandable device prepared from a mixture comprising a polysaccharide and locust bean gum, the device being compressed to form a compressed device suitably sized for swallowing, the compressed device having a coating erodible by gastric fluid applied to an outer surface thereof or being housed within an ingestible capsule erodible by gastric fluid.

87. The method according to claim 86 where the gastric retention device comprises a compressed device that, upon ingestion, expands sufficiently, and is sufficiently robust upon expansion, to preclude passage of the device through a subject's pylorus for a predetermined time up to at least 24 hours while still allowing food to pass, the compressed device further comprising a material selected from the group consisting of therapeutics, diagnostics, plasticizers, pH adjusters, GI motility adjusters, viscosity adjusters, expansion agents, surfactants, and mixtures thereof, the compressed device having a coating erodible by gastric fluid applied to an outer surface thereof or being housed in a capsule erodible by gastric fluid.

88. The method according to claim 83 where the gastric retention device comprises an expandable device prepared from a mixture comprising xanthan gum and locust bean gum, the device being compressed to form a compressed device, the compressed device having a coated applied to an outer surface thereof or being housed in a capsule erodible by gastric fluid.

89. The method of claim 84 where the GRD is of a size sufficient to pass through a pylorus and provides delivery of the diagnostic and/or therapeutic to the colon.

90. The method of claim 84 where the GRD further comprises an enteric coating and provides delivery of the diagnostic and/or therapeutic to the colon.

91. A method of appetite suppression, comprising:
providing a gastric retention device that expands sufficiently in the stomach of a subject to at least partially suppress appetite in the subject; and
administering the gastric retention device to the subject.

92. The method of claim 91 where the device further comprises an effective amount of a fatty acid, an appetite suppressant, a weight loss agent, or combinations thereof.

93. A method of appetite suppression, comprising:
providing a gastric retention device that expands sufficiently in the intestine of a subject to at least partially suppress appetite in the subject; and
administering the gastric retention device to the subject.

94. The method of claim 93 where the device further comprises an effective amount of a fatty acid, an appetite suppressant, a weight loss agent, or combinations thereof.

95. A dosage form comprising a dehydrated polymer gel formed to a size suitable for swallowing and having an excipient, the dehydrated polymer having a weight of about 1.2 grams or less.

96. The dosage form of claim 95 formed to a size suitable for nasal administration.

97. The dosage form of claim 95 formed to a size suitable for vaginal administration.

98. The dosage form of claim 95 formed to a size suitable for rectal administration.

99. The dosage form of claim 95 formed to a size suitable for intestinal administration.

100. The dosage form of claim 95 formed to a size suitable for insertion into a wound.

101. The method according to claim 83, further comprising a diagnostic or therapeutic agent, where delivery of the agent at two hours ranges from about 2% to about 70% of the total agent available for delivery, and delivery of the agent at twenty four hours ranges from about 35% to about 100% of the total diagnostic or therapeutic available for delivery.

102. The method according to claim 83, further comprising ranitidine HCl, where delivery is measured in vitro in a USP paddle stirring apparatus in appropriate aqueous media at 37° C., and where delivery of the ranitidine HCl at two hours is up to about 70% of the total ranitidine HCl available for delivery, and delivery of the ranitidine HCl at twenty four hours is about 100% of the total ranitidine HCl available for delivery.

103. The method according to claim 83, further comprising riboflavin, where delivery is measured in vitro in a USP paddle stirring apparatus in appropriate aqueous media at 37° C., and where delivery of the riboflavin at two hours is up to about 2% of the total riboflavin available for delivery, and delivery of the riboflavin at twenty four hours is up to about 70% of the total riboflavin available for delivery.

104. The method according to claim 83, where the diagnostic or therapeutic is riboflavin, where delivery is measured in vivo as urinary excretion of riboflavin, and where delivery of the riboflavin at two hours is up to about 15% of the total riboflavin available for delivery, and delivery of the riboflavin at twenty four hours is about 100% of the total riboflavin available for delivery.

105. The method according to claim 83, where the diagnostic or therapeutic is hydrochlorothiazide and hydrochlorothiazide delivery is assessed by determining urine output, and where urine output at two hours is about 10% of the total 42 hour urine output, and urine output at twenty four hours is about 75% of the total 42 hour urine output.

106. A method of using the gastric retention device of claim 83, where administering the diagnostic or therapeutic in the gastric retention device produces a first result which, when compared to a second result obtained by administering the diagnostic or therapeutic without the gastric retention device, produces a desired biological benefit.

107. The method of claim 106, where the diagnostic or therapeutic is hydrochlorothiazide and the desired biological benefit is increased total urine output.

108. The method of claim 83 for determining a GI absorption site of a diagnostic or therapeutic, where administration comprises administering a GRD of sufficient size to prevent passage of the GRD through a pylorus, and further comprising determining the GI absorption site of the diagnostic or therapeutic.

109. The method of claim 83 for determining a GI absorption site of a diagnostic or therapeutic, where administration comprises administering a GRD of sufficient size to pass through a pylorus, and further comprising determining the GI absorption site of the diagnostic or therapeutic.

110. The dosage form according to claim 100, wherein the combined weight of the excipient and the dehydrated polymer is less than about 1.2 grams.

111. The dosage form according to claim 110, further comprising a diagnostic or therapeutic agent.

112. The dosage form according to claim 111, wherein the dosage form comprises a therapeutic agent.

113. The dosage form according to claim 112, wherein the excipient, therapeutic agent and dehydrated polymer have a combined weight of less than about 1.2 grams.

114. The dosage form according to claim 113, wherein the combined weight is less than about 1 gram.

115. The dosage form according to claim 114, wherein the combined weight is less than about 0.8 gram.

116. The dosage form according to claim 95, further comprising a lipid material.

117. The dosage form according to claim 116, wherein the lipid material is an oil.

118. The dosage form according to claim 116, wherein the lipid material is a vegetable oil.

119. The dosage form according to claim 116, wherein the lipid material is a fatty acid.

120. A dosage form, comprising a dehydrated polymer gel and an excipient, formed to a size suitable for vaginal administration, wherein the weight of the dehydrated polymer is less than about 10 grams.

121. The dosage form according to claim 120, wherein the combined weight of the dehydrated polymer and the excipient is less than about 10 grams.

122. The dosage form according to claim 121, wherein the dosage includes a therapeutic agent, the combined weight of the dehydrated polymer, excipient and therapeutic agent being less than about 10 grams.

123. A method for making a gastric retention device, comprising:
forming a mixture comprising a swellable polymeric gel;
processing the mixture to form a dried gel having a roughly parallepiped shape; and;
compressing the dried gel to form a compressed gel having a size suitable for oral administration.

124. The method according to claim 123, wherein forming the mixture comprises mixing a therapeutic agent, an excipient and a polymeric material.

125. The method according to claim 124, wherein the polymeric material is a polysaccharide.

126. The method according to claim 123, wherein processing the mixture to form a dried gel comprises freeze drying.

127. The method according to claim 123, wherein processing the mixture to form a dried gel comprises vacuum drying at elevated temperature.

128. The method according to claim 123, wherein the dried gel is compressed to a volume of from about 0.3 mL to about 1.4 mL.

129. The method according to claim 128, wherein the dried gel is compressed to a volume of from about 0.3 mL to about 1.1 mL.

130. The method according to claim 123, further comprising coating the compressed gel with an erodible coating.

131. The method according to claim 123, further comprising placing the compressed gel into a capsule.

132. The method according to claim 123, wherein the dried gel weighs less than about 1.2 grams.

133. The gastric retention device of claim 95 where the diagnostic or therapeutic agent is abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, captopril, carvedilol, caspoflugin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fexofenadine, fluticasone propionate, furosemide, gancyclovir, hydrochlorothiazide/triamterene, lamivudine, lamotrigine, lithium carbonate, losartan potassium, melphalan, mercaptopurine, mesalazine, metformin, methyldopa, minocycline, mupirocin calcium cream, nabumetone, naratriptan, omeprazole, ondansetron hydrochloride, orlistat (or a pharmaceutically acceptable salt thereof), ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, selegiline, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine, lamivudine or combinations thereof.

134. The method of claim 83 where the therapeutic or diagnostic is abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, carvedilol, caspofingin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fluticasone propionate, furosemide, hydrochlorothiazide/triamterene, lamivudine, lamotrigine, lithium carbonate, losartan potassium, melphalan, mercaptopurine, mesalazine, mupirocin calcium cream, nabumetone, naratriptan, omeprazole, ondansetron hydrochloride, ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine or lamivudine, or mixtures thereof.

135. The gastric retention device according to claim 1, further comprising a lipid material.

136. The gastric retention device according to claim 135, wherein the lipid material is a fatty acid.

137. The gastric retention device according to claim 136, wherein the fatty acid is sodium myristate.

138. The gastric retention device according to claim 135, wherein the lipid material is a vegetable oil.

139. The gastric retention device according to claim 135, wherein the lipid material is present in an amount effective to decrease the rate of gastric emptying.

140. The method according to claim 69, wherein processing comprises inducing gelation at about room temperature.

141. The method according to claim 83, wherein administering the device to a subject reduces the subject's appetite.

142. The method according to claim 141, wherein the gastric retention device comprises a lipid material, an appetite suppressant, a weight loss agent or combinations thereof.

143. The method according to claim 83, further comprising riboflavin, where delivery is measured in vitro in a USP paddle stirring apparatus in appropriate aqueous media at 37° C., and where delivery of the riboflavin at two hours is up to about 2% of the total riboflavin available for delivery, and delivery of the riboflavin at twenty four hours is up to about 35% of the total riboflavin available for delivery.

144. The method according to claim 83, further comprising riboflavin, where delivery is measured in vitro in a USP paddle stirring apparatus in appropriate aqueous media at 37° C., and where delivery of the riboflavin at two hours is up to about 30% of the total riboflavin available for delivery, and delivery of the riboflavin at twenty four hours is up to at least 75% of the total riboflavin available for delivery.
